(19) United States
(12) Patent Application Publication
(10) Pub. No.: US 2025/0391502 A1
(43) Pub. Date: Dec. 25, 2025

(54) METHODS AND SYSTEMS FOR DETERMINING ANEUPLOIDY-BASED INTRATUMOR HETEROGENEITY

(71) Applicant: Foundation Medicine, Inc., Boston, MA (US)

(72) Inventors: Saumya Dushyant SISOUDIYA, Rockville, MD (US); Smruthy K. SIVAKUMAR, Everett, MA (US); Ethan S. SOKOL, Somerville, MA (US); Meagan K. MONTESION, Somerville, MA (US)

(73) Assignee: Foundation Medicine, Inc., Boston, MA (US)

(21) Appl. No.: 19/242,695

(22) Filed: Jun. 18, 2025

Related U.S. Application Data
(60) Provisional application No. 63/662,206, filed on Jun. 20, 2024.

(51) Int. Cl. G16B 20/10; G16B 30/00; G16H 50/30

(52) U.S. Cl. CPC G16B 20/10; G16B 30/00; G16H 50/30

(57) ABSTRACT

Methods for characterizing aneuploidy based intratumor heterogeneity for a tumor of a tumor type for a subject. The methods may comprise, for example, obtaining sample aneuploidy data for the tumor, calling subclonal aneuploidy events in the sample aneuploidy data and generating an intratumor heterogeneity score for the tumor sample, and use of the aneuploidy based intratumor heterogeneity in cancer treatment and prognostics.

### CROSS-REFERENCE TO RELATED APPLICATIONS

This application claims the priority benefit of U.S. Provisional Application No. 63/662,206 filed Jun. 20, 2024, the entire contents of which is incorporated herein by reference for all purposes.

### FIELD

The present disclosure relates to methods and systems for determining a measure of aneuploidy based intratumor heterogeneity and use of the aneuploidy based intratumor heterogeneity in cancer treatment and prognostics.

### BACKGROUND

Intratumor heterogeneity (ITH) is a complex biomarker that quantifies intratumor variation in the genome of a sample from a subject that may be associated with disease progression and therapy resistance. Aneuploidy, arising from the abnormal number of chromosomal copies, is a common hallmark in tumors and is often a key driver of ITH.

Identification aneuploidy-based ITH of sample from a subject (e.g., a patient) can help understand tumor evolution and guide treatment determination. Additionally, certain cancers, (e.g., ovarian cancer) are mostly aneuploidy driven, making more important to be able to assess aneuploidy-based ITH. Determination of aneuploidy-based ITH rely on analysis that are often not available using current methods utilizing short variants. Thus, improved methods for determining aneuploidy-based ITH are required to improve the predictive accuracy of this aneuploidy-based ITH and associated healthcare outcomes.

### BRIEF SUMMARY

Disclosed herein are methods and systems for determining significantly clonal and subclonal aneuploidy events in cancer to generate a patient specific aneuploidy-based ITH metric. The methods provided herein, take advantage of longitudinal biopsies to identify aneuploidy events that may be indicative of heterogeneity in a specific cancer type. The methods will then allow for prediction of aneuploidy-based ITH in subjects with the same cancer type who have only received one biopsy. Further, aneuploidy-based ITH can then be used to identify tumors likely to quickly gain resistance to targeted therapies. The aneuploidy-based ITH can also be combined with other ITH metrics, such as but not limited to structural variant ITH, point mutation ITH or mRNA based ITH, to increase their descriptive and diagnostic power. Also provided herein are methods for generating an aneuploidy-based ITH metric for non-small cell lung cancer tumors and ovarian cancer tumors. The metrics can be generated using a single sample and related to cancer prognosis, resistance to cancer therapies, and outcomes.

In some instances, provided herein are methods of characterizing aneuploidy based intratumor heterogeneity for a tumor of a tumor type from a subject, comprising: obtaining sample aneuploidy data for the tumor; calling subclonal aneuploidy events in the sample aneuploidy data, wherein a subject aneuploidy event is characterized as subclonal based on a comparison of the subject aneuploidy event to a corresponding reference aneuploidy event for the tumor type, wherein the reference aneuploidy event had been characterized by: obtaining reference aneuploidy data for a plurality of reference tumor samples of the tumor type, wherein the plurality of reference tumor samples comprises at least two tumor samples obtained at different time points from each reference subject in a plurality of reference subjects, characterizing, for each aneuploidy event in a plurality of aneuploidy events, as unique or shared among the at least two tumor samples from each reference subject in the plurality of reference subjects, determining, for each aneuploidy event in the plurality of aneuploidy events, whether uniqueness of the aneuploidy event within the plurality of aneuploid events is enriched compared to uniqueness of all aneuploidy events within the plurality of aneuploidy events, and characterizing the aneuploidy event as significantly subclonal or significantly clonal based on enrichment of the uniqueness of the aneuploidy event; and generating an intratumor heterogeneity score for the tumor sample based on a number of called significantly subclonal events in the sample aneuploidy data for the tumor sample.

In some instances, provided herein are methods of characterizing significantly subclonal events or significantly clonal events of a tumor type, comprising: obtaining reference aneuploidy data for a plurality of reference tumor samples of the tumor type, wherein the plurality of reference tumor samples comprises at least two tumor samples obtained at different time points from each reference subject in a plurality of reference subjects, characterizing, for each aneuploidy event in a plurality of aneuploidy events, as unique or shared among the at least two tumor samples from each reference subject in the plurality of reference subjects, determining, for each aneuploidy event in the plurality of aneuploidy events, whether uniqueness of the aneuploidy event within the plurality of aneuploid events is enriched compared to uniqueness of all aneuploidy events within the plurality of aneuploidy events, and characterizing the aneuploidy event as significantly subclonal or significantly clonal based on enrichment of the uniqueness of the aneuploidy event.

In some instances, the sample aneuploidy data comprises one or more aneuploidy event annotations detected in a single sample collected from the tumor. In some instances, the reference aneuploidy data comprises one or more aneuploidy event annotations for the plurality of reference tumor samples of the tumor type. In some instances, the one or more aneuploidy event annotations are characterized as a variation in chromosome number from a base ploidy of the sample.

In some instances, the one or more aneuploidy event annotations comprise a plurality of aneuploidy events. In some instances, an aneuploidy event in the plurality of aneuploidy events is a gain of a chromosomal portion or a loss of a chromosomal portion. In some instances, a chromosomal portion is a chromosomal arm. In some instances, a chromosomal portion is a cytoband.

In some instances, the tumor type is non-small cell lung cancer (NSCLC), breast cancer, or ovarian cancer. In some instances, the tumor type is a B cell cancer (multiple myeloma), a melanoma, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor. In some instances, the tumor type comprises the tumor type comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some instances, determining whether uniqueness of an aneuploidy event within the plurality of aneuploid events is enriched compared to uniqueness of all aneuploidy events within the plurality of aneuploidy events comprises comparing how often the aneuploidy event is subclonal, performing a Fisher's exact test, or performing a chi-squared test.

In some instances, performing the Fisher's exact test comprises generating an odds ratio. In some instances, an aneuploidy event is significantly subclonal if the fold change in odds ratio is beyond a cutoff in the negative direction. In some instances, the cutoff is more negative than about-1.5. In some instances, an aneuploidy event is significantly clonal if the fold change in odds ratio beyond a cutoff in the positive direction. In some instances, the cutoff is greater than about 1.5.

In some instances, obtaining sample aneuploidy data comprises; performing a tumor biopsy; extracting tumor nucleic acids; sequencing, by a sequencer, the extracted tumor nucleic acids; receiving a tumor sequence data from the sequencers; and providing the tumor sequence data to a program configured to receive tumor sequence data and identify a plurality of aneuploidy annotations. In some instances, the tumor biopsy is a liquid biopsy and comprises cell-free DNA (cfDNA), circulating tumor DNA (ctDNA), RNA or any combination thereof. In some instances, the liquid biopsy comprises blood, plasma, cerebrospinal fluid, sputum, stool, urine, or saliva. In some instances, extracting tumor nucleic acids comprises extracting ctDNA.

In some instances, the sequencing comprises use of a massively parallel sequencing (MPS) technique, whole genome sequencing (WGS), RNA sequencing (RNAseq), low pass sequencing, whole exome sequencing, targeted sequencing, direct sequencing, or Sanger sequencing technique. In some instances, the sequencing comprises massively parallel sequencing, and the massively parallel sequencing technique comprises next generation sequencing (NGS). In some instances, the sequencer comprises a next generation sequencer.

In some instances, generating the intratumor heterogeneity score comprises summing the number of called significantly subclonal events in the sample aneuploidy event data for the tumor sample. In some instances, the methods further comprise generating an intratumor heterogeneity indicator, wherein the intratumor heterogeneity indicator relates to the relationship between the intratumor heterogeneity score and a determined threshold. In some instances, the determined threshold comprises an upper threshold and a lower threshold. In some instances, the intratumor heterogeneity indicator is high if the intratumor heterogeneity metric is greater than or equal to the upper threshold, the intratumor heterogeneity indicator is intermediate if the intratumor heterogeneity metric is greater than the lower threshold and less than the lower threshold, and the intratumor heterogeneity indicator is low if intratumor heterogeneity metric is less than or equal to the lower threshold. In some instances, the high intratumor heterogeneity score relates to poor prognosis, quick resistance to cancer therapies, or poor outcomes.

In some instances, the methods further comprise generating an aneuploidy burden score by integrating the intratumor heterogeneity score with digital pathology-based heterogeneity, single cell heterogeneity scores, radiological heterogeneity scores, aneuploidy burden, cytoband features, CN segment features for the tumor.

Also provided herein are methods of selecting or treatment for an individual with cancer, comprising (a) characterizing aneuploidy based intratumor heterogeneity in a sample from the individual according to the methods of any of the methods described herein, and (b) selecting a treatment based on the aneuploidy based intratumor heterogeneity.

Also provided herein are methods of treating or delaying progression of cancer in an individual, comprising: (a) characterizing aneuploidy based intratumor heterogeneity in a sample from the individual according to any of the methods described herein; and (b) administering to the individual an effective amount of a therapy based on the intratumor heterogeneity.

Also provided herein are methods of predicting survival of an individual having cancer, comprising acquiring knowledge of an intratumor heterogeneity indicator in a sample from the individual, wherein responsive to the acquisition of said knowledge, the individual is predicted to have longer survival when the intratumor heterogeneity indicator is low than if the intratumor heterogeneity indicator is high. In some aspects, acquiring knowledge of an intratumor heterogeneity indicators comprises generating an intratumor heterogeneity indicator according to any of the methods described herein.

Also provided herein are methods comprising: obtaining sample aneuploidy data for a non-small cell lung cancer (NSCLC) tumor of a subject; calling subclonal aneuploidy events in the sample aneuploidy data, wherein a subject aneuploidy event is characterized as subclonal based on a comparison of the subject aneuploidy event to a corresponding NSCLC reference aneuploidy event on a list of NSCLC significantly subclonal events; and generating an intratumor heterogeneity score based on a number of aneuploidy events in the aneuploidy event data on the list of NSCLC significantly subclonal events.

In some instances, the list of NSCLC significantly subclonal events comprise a plurality of NSCLC reference aneuploidy events. In some instances, the plurality of NSCLC reference aneuploidy events comprise arm level chromosome gains of 2p, 2q, 3p, 4q, 6q, 10q, 12q, 13q, 15q, 16q, 17p, 18q, 19p, 21q, and 22q, and arm level chromosomal losses of 1q, 2p, 2q, 3q, 5p, 6p, 7p, 7q, 11p, 11q, 12q, 16p, 17q, and 20q.

In some instances, the sample aneuploidy data comprises one or more aneuploidy event annotations. In some instances, the one or more aneuploidy event annotations are characterized as a variation in chromosome number from a base ploidy of the sample. In some instances, the one or more aneuploidy event annotations comprise a plurality of aneuploidy events. In some instances, an aneuploidy event in the plurality of aneuploidy events is an arm level chromosome gain or an arm level chromosomal loss.

In some instances, generating the intratumor heterogeneity score comprises summing the number of called significantly subclonal events in the sample aneuploidy event data for the tumor sample. In some instances, the methods further comprise generating an intratumor heterogeneity indicator, wherein the intratumor heterogeneity indicator relates to the relationship between the intratumor heterogeneity score and a determined threshold. In some instances, a determined threshold comprises an upper threshold and a lower threshold. In some instances, the intratumor heterogeneity indicator is high if the intratumor heterogeneity metric is greater or equal to the upper threshold, the intratumor heterogeneity indicator is intermediate if the intratumor heterogeneity metric is greater than the lower threshold and less than the lower threshold, and the intratumor heterogeneity indicator is low if intratumor heterogeneity metric is less than or equal to the lower threshold.

In some instances, the upper threshold is between 2 and 6. In some instances, the lower threshold is between 0 and 2. In some instances, the upper threshold is 4 and the lower threshold is 1.

In some instances, a high intratumor heterogeneity score relates to poor prognosis, quick resistance to cancer therapies, or poor outcomes.

In some instances, obtaining sample aneuploidy data comprises; performing a tumor biopsy; extracting tumor nucleic acids; sequencing, by a sequencer, the extracted tumor nucleic acids; receiving a tumor sequence data from the sequencers; and providing the tumor sequence data to a program configured to receive tumor sequence data and identify a plurality of aneuploidy annotations. In some instances, the tumor biopsy is a liquid biopsy and comprises cell-free DNA (cfDNA), circulating tumor DNA (ctDNA), RNA or any combination thereof. In some instances, the sample is a liquid biopsy sample and comprises blood, plasma, cerebrospinal fluid, sputum, stool, urine, or saliva. In some instances, extracting tumor nucleic acids comprises extracting ctDNA.

In some instances, the sequencing comprises use of a massively parallel sequencing (MPS) technique, RNA sequencing (RNAseq), low pass sequencing, whole genome sequencing (WGS), whole exome sequencing, targeted sequencing, direct sequencing, or Sanger sequencing technique. In some instances, the sequencing comprises massively parallel sequencing, and the massively parallel sequencing technique comprises next generation sequencing (NGS). In some instances, the sequencer comprises a next generation sequencer.

Also provided herein are methods comprising: obtaining sample aneuploidy data for an ovarian tumor of a subject; calling subclonal events in the sample aneuploidy data, wherein a subject aneuploidy event is characterized as subclonal based on a comparison of the subject aneuploidy event to a corresponding ovarian reference aneuploidy event on a list of ovarian significantly subclonal events; and generating an intratumor heterogeneity score based on a number of aneuploidy events in the aneuploidy event data on a list of ovarian significantly subclonal events.

In some instances, the list of ovarian significantly subclonal events comprise a plurality of ovarian reference aneuploidy events. In some instances, the plurality of ovarian reference aneuploidy events comprise arm level chromosome gains of 1p, 3p, 4q, 11p, 12q, 13q, 16p, 16q, 17q, 19q, 21q and 22q, and arm level chromosomal losses of 1q, 2p, 2q, 3p, 5p, 6p, 7q, 8q, 10p, 12q, 17q, 20p, 20q, and 21q.

In some instances, the sample aneuploidy data comprises one or more aneuploidy event annotations. In some instances, the reference aneuploidy data comprises one or more aneuploidy event annotations for the plurality of reference ovarian tumor samples. In some instances, the one or more aneuploidy event annotations are characterized as a variation in chromosome number from a base ploidy of the sample.

In some instances, the one or more aneuploidy event annotations comprise a plurality of aneuploidy events. In some instances, an aneuploidy event in the plurality of aneuploidy events is an arm level chromosome gain or an arm level chromosomal loss.

In some instances, generating the intratumor heterogeneity score comprises summing the number of called significantly subclonal events in the sample aneuploidy event data for the tumor sample. In some instances, the methods further comprise generating an intratumor heterogeneity indicator, wherein the intratumor heterogeneity indicator relates to the relationship between the intratumor heterogeneity score and a determined threshold. In some instances, a determined threshold comprises an upper threshold and a lower threshold. In some instances, the intratumor heterogeneity indicator is high if the intratumor heterogeneity metric is greater than or equal to the upper threshold, the intratumor heterogeneity indicator is intermediate if the intratumor heterogeneity metric is greater than the lower threshold and less than the lower threshold, and the intratumor heterogeneity indicator is low if intratumor heterogeneity metric is less than or equal to the lower threshold.

In some instances, the upper threshold is between 2 and 6. In some instances, the lower threshold is between 0 and 2. In some instances, the upper threshold is 4 and the lower threshold is 1.

In some instances, a high intratumor heterogeneity score relates to poor prognosis, quick resistance to cancer therapies, or poor outcomes.

In some instances, obtaining sample aneuploidy data comprises; performing a tumor biopsy; extracting tumor nucleic acids; sequencing, by a sequencer, the extracted tumor nucleic acids; receiving a tumor sequence data from the sequencers; and providing the tumor sequence data to a program configured to receive tumor sequence data and identify a plurality of aneuploidy annotations. In some instances, the tumor biopsy is a liquid biopsy and comprises cell-free DNA (cfDNA), circulating tumor DNA (ctDNA), RNA or any combination thereof. In some instances, the sample is a liquid biopsy sample and comprises blood, plasma, cerebrospinal fluid, sputum, stool, urine, or saliva. In some instances, extracting tumor nucleic acids comprises extracting ctDNA.

In some instances, the sequencing comprises use of a massively parallel sequencing (MPS) technique, RNA sequencing (RNAseq), low pass sequencing, whole genome sequencing (WGS), whole exome sequencing, targeted sequencing, direct sequencing, or Sanger sequencing technique. In some instances, the sequencing comprises massively parallel sequencing, and the massively parallel sequencing technique comprises next generation sequencing (NGS). In some instances, the sequencer comprises a next generation sequencer.

Also provided herein are methods, comprising: obtaining sample aneuploidy data for a breast tumor of a subject; calling subclonal events in the sample aneuploidy data, wherein a subject aneuploidy event is characterized as subclonal based on a comparison of the subject aneuploidy event to a corresponding breast cancer reference aneuploidy event on a list of breast cancer significantly subclonal events; and generating an intratumor heterogeneity score based on a number of aneuploidy events in the aneuploidy event data on a list of breast cancer significantly subclonal events.

In some aspects the list of breast cancer significantly subclonal events comprise a plurality of breast cancer reference aneuploidy events. In some aspects, the plurality of breast cancer reference aneuploidy events comprise arm level chromosome gains of 1p, 2p, 2q, 3p, 4p, 4q, 9q, 10q, 11p, 11q, 13q, 14q, 15q, 16q, 17p, 18p, 18q, 19p, 19q, 21q and 22q, and arm level chromosomal losses of 1p, 2p, 2q, 3q, 5p, 6p, 7p, 8q, 10p, 16p, 19p, 19q, 20p, and 20q.

In some aspects, the sample aneuploidy data comprises one or more aneuploidy event annotations. In some aspects, the reference aneuploidy data comprises one or more aneuploidy event annotations for the plurality of reference breast tumor samples. In some aspects, the one or more aneuploidy event annotations are characterized as a variation in chromosome number from a base ploidy of the sample.

In some aspects, the one or more aneuploidy event annotations comprise a plurality of aneuploidy events. In some aspects, an aneuploidy event in the plurality of aneuploidy events is an arm level chromosome gain or an arm level chromosomal loss.

In some aspects, generating the intratumor heterogeneity score comprises summing the number of called significantly subclonal events in the sample aneuploidy event data for the breast tumor sample.

In some aspects, the methods further comprising generating an intratumor heterogeneity indicator, wherein the intratumor heterogeneity indicator relates to the relationship between the intratumor heterogeneity score and a determined threshold. In some aspects, determined threshold comprises an upper threshold and a lower threshold. In some aspects, the intratumor heterogeneity indicator is high if the intratumor heterogeneity metric is greater than or equal to the upper threshold, the intratumor heterogeneity indicator is intermediate if the intratumor heterogeneity metric is greater than the lower threshold and less than the lower threshold, and the intratumor heterogeneity indicator is low if intratumor heterogeneity metric is less than or equal to the lower threshold. In some aspects, the upper threshold is between 1 and 5. In some aspects, the lower threshold is between 0 and 1. In some aspects, the upper threshold is 1 and the lower threshold is 5.

In some aspects, a high intratumor heterogeneity score relates to poor prognosis, quick resistance to cancer therapies, or poor outcomes. In some aspects, the poor outcomes comprises a shorter survival. In some aspects, the survival is progression free survival.

In some aspects, the methods comprise obtaining sample aneuploidy data comprises; performing a tumor biopsy; extracting tumor nucleic acids; sequencing, by a sequencer, the extracted tumor nucleic acids; receiving a tumor sequence data from the sequencers; and providing the tumor sequence data to a program configured to receive tumor sequence data and identify a plurality of aneuploidy annotations. In some aspects, the tumor biopsy is a liquid biopsy and comprises cell-free DNA (cfDNA), circulating tumor DNA (ctDNA), RNA, or any combination thereof. In some aspects, the sample is a liquid biopsy sample and comprises blood, plasma, cerebrospinal fluid, sputum, stool, urine, or saliva. In some aspects, extracting tumor nucleic acids comprises extracting ctDNA.

In some aspects, the sequencing comprises use of a massively parallel sequencing (MPS) technique, whole genome sequencing (WGS), RNA sequencing (RNAseq), low pass sequencing, whole exome sequencing, targeted sequencing, direct sequencing, or Sanger sequencing technique. In some aspects, the sequencing comprises massively parallel sequencing, and the massively parallel sequencing technique comprises next generation sequencing (NGS). In some aspects, the sequencer comprises a next generation sequencer.

In some aspects, subject is diagnosed with stage 1 breast cancer. In some aspects, the subject received a breast cancer therapy. In some aspects, the breast cancer therapy comprises a CDK4/6 inhibitor and an endocrine therapy in a first-line metastatic setting.

Also provided herein are method of predicting survival of an individual having breast cancer, comprising acquiring knowledge of an intratumor heterogeneity indicator in a sample from the individual, wherein responsive to the acquisition of said knowledge, the individual is predicted to have longer survival when the intratumor heterogeneity indicator is low than if the intratumor heterogeneity indicator is high. In some aspects, acquiring knowledge of an intratumor heterogeneity indicators comprises generating an intratumor heterogeneity indicator according to the methods described herein.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 provides a non-limiting example of a method for characterizing significantly subclonal events or significantly clonal events of a defined tumor type.

FIG. 2 provides a non-limiting example of a method for characterizing aneuploidy based intratumor heterogeneity for tumor of a tumor type from a subject.

FIG. 3 depicts an exemplary computing device or system in accordance with one embodiment of the present disclosure.

FIG. 4 depicts an exemplary computer system or computer network, in accordance with some instances of the systems described herein.

FIGS. 5A-5B provide non-limiting examples of a plot of p-value vs odds ratio (OR) for NSCLC aneuploidy events colored by significance and unique/shared enrichment. FIG. 5A shows arm gains identified in NSCLC tumors. FIG. 5B shows arm losses identified in NSCLC tumors.

FIG. 6 provides a non-limiting example of a histogram of the number of significantly subclonal events in NSCLC samples.

FIG. 7 provides a non-limiting example of a bar plot of intratumor heterogeneity (ITH) scores in NSCLC by time between biopsies.

FIG. 8 provides a non-limiting example of a bar plot of ITH scores in NSCLC by tumor stage at biopsy.

FIGS. 9A-9B provides non-limiting examples of a plot of p-value vs odds ratio (OR) for ovarian cancer aneuploidy events colored by significance and sharing. FIG. 9A shows arm gains identified in ovarian tumors. FIG. 9B shows arm losses identified in ovarian tumors shaded by significant and unique/shared enrichment.

FIG. 10 provides a non-limiting example of a histogram of the number of significantly subclonal events in ovarian cancer samples.

FIGS. 11A-11B provides a non-limiting example of a bar plots of aneuploidy associated ITH in ovarian cancer. FIG. 11A shows ITH scores by time between biopsies for the ovarian cancer samples. FIG. 11B shows ITH scores by tumor stage of the ovarian cancer samples.

FIG. 12 provides a non-limiting example of a hazard plot for progression-free survival for stage 1 at diagnosis breast cancer patients separated by aneuploidy associated ITH scores. The dotted lines represent the time in months wherein 50% of the group exhibited progression free survival.

### DETAILED DESCRIPTION

Disclosed herein are method and systems that can allow for the characterization of aneuploidy based intratumor heterogeneity in a tumor with a single sample collected from a patient. Because aneuploidy-based ITH is a measure of variation in a tumor, traditional methods to characterized ITH rely on identifying aneuploidy events, such as chromosomal arm gains and losses in data from multiple tumor samples collected with spatially or temporally distinct biopsies. Thus, as described, the methods and systems confer a technical advantage of eliminating the need for spatially or temporally distinct biopsies in determining aneuploidy-based ITH.

The methods and systems described herein, can be used to take advantage of aneuploidy event data from longitudinal samples in distinct cancer types to identify regions of the genome likely to contribute to aneuploidy-based ITH for that cancer type. Once the regions of the genome that contribute to aneuploidy-based ITH are identified for a cancer type, methods described herein can be used to characterize aneuploidy-based ITH using aneuploidy events identified in a single sample of the tumor. As such, the methods and systems described herein improve cancer diagnostics and therapy by making the characterization of aneuploidy-based ITH more efficient than with previously disclosed methods.

Methods and systems for characterizing aneuploidy based intratumor heterogeneity (ITH) in tumors are described. The methods and systems take advantage of matched longitudinal samples from the tumor type to determine the chromosome portions that contribute to aneuploidy-based ITH in that tumor type. Once the chromosome portions contributing to aneuploidy-based ITH in a tumor type are determined, an aneuploidy-based ITH metric can be generated using a sample (e.g., a single sample) from a patient with that tumor type. The methods and systems described herein remove the need for spatially distinct biopsies or biopsies collected at multiple time points to understand aneuploidy-based ITH for each patient in the clinic. The methods and systems will allow health care providers to obtain and consider aneuploidy-based ITH without needing to spend the time and money to sample a tumor at multiple time points or sample multiple locations on a single tumor. The methods and systems in turn provide benefits to patients because aneuploidy-based ITH metrics that are associated with prognosis, resistance to targeted therapies, and overall outcomes.

In some instances, for example, methods are described that comprise obtaining aneuploidy data for a tumor from a patient, identifying subclonal aneuploidy events by comparing the aneuploidy events to a reference with significantly clonal and significantly subclonal aneuploidy events in the same tumor type. In some instances, the significantly subclonal or significantly clonal aneuploidy events for a tumor type have been determined using a characterization of unique or shared aneuploidy events between multiple tumor samples from the same patient for a plurality of patients with the same tumor type and determining the enrichment of the uniqueness of each aneuploidy event compared to the uniqueness of all other aneuploidy events. In some instances, the methods described herein comprise generating an intratumor heterogeneity (ITH) score for the tumor sample based on the number of significantly subclonal events identified in the tumor sample. In some instances, the described method are used to generate an ITH score for a NSCLC tumor or an ovarian cancer tumor.

In some instances, the aneuploidy data comprises on or more aneuploidy events, comprising one or more aneuploidy event annotation. In some instances, an aneuploidy event annotation is characterized as a variation in chromosome number from a base ploidy of the sample and the event may be a gain or loss of a portion of a chromosome such as a chromosome arm or cytoband. In some instances, an aneuploidy event annotation is characterized as a variation in chromosome number of 2 for an autosome.

In some instances, the uniqueness of an aneuploidy event compared to the uniqueness of other aneuploidy events is determined using an enrichment analysis such as a Fisher's exact test. In some instances, an aneuploidy event is significantly clonal or significantly subclonal based on the odds ratio (OR) resulting from the enrichment analysis. In some instances, an ITH score can be compared to a determined threshold in order to generate and intratumor heterogeneity indicator. In some instances, the significantly subclonal events for NSCLC tumors are arm level chromosome gains of 2p, 2q, 3p, 4q, 6q, 10q, 12q, 13q, 15q, 16q, 17p, 18q, 19p, 21q, and 22q, and arm level chromosomal losses of 1q, 2p, 2q, 3q, 5p, 6p, 7p, 7q, 11p, 11q, 12q, 16p, 17q, and 20q. In some instances, the significantly subclonal events for ovarian cancer tumors are ovarian reference aneuploidy events comprise arm level chromosome gains of 1p, 3p, 4q, 11p, 12q, 13q, 16p, 16q, 17q, 19q, 21q and 22q, and arm level chromosomal losses of 1q, 2p, 2q, 3p, 5p, 6p, 7q, 8q, 10p, 12q, 17q, 20p, 20q, and 21q.

### Definitions

Unless otherwise defined, technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”).

As used herein, the term “ploidy” refers to the average copy number for a plurality of gene loci in a tumor sample. In some instances, the “ploidy” of a tumor sample may differ from the number of complete sets of chromosomes in a cell, and hence the number of possible alleles for autosomal genes (i.e., genes located on numbered, non-sexual chromosomes), due to heterogeneity of the tumor sample (i.e., the variation in tumor sample purity).

When a range of values is provided, it is to be understood that each intervening value between the upper and lower limit of that range, and any other stated or intervening value in that states range, is encompassed within the scope of the present disclosure. Where the stated range includes upper or lower limits, ranges excluding either of those included limits are also included in the present disclosure.

Some of the analytical methods described herein include mapping sequences to a reference sequence, determining sequence information, and/or analyzing sequence information. It is well understood in the art that complementary sequences can be readily determined and/or analyzed, and that the description provided herein encompasses analytical methods performed in reference to a complementary sequence.

The section headings used herein are for organization purposes only and are not to be construed as limiting the subject matter described. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

The figures illustrate processes according to various embodiments. In the exemplary processes, some blocks are, optionally, combined, the order of some blocks is, optionally, changed, and some blocks are, optionally, omitted. In some examples, additional steps may be performed in combination with the exemplary processes. Accordingly, the operations as illustrated (and described in greater detail below) are exemplary by nature and, as such, should not be viewed as limiting.

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### Methods for Characterizing of Characterizing Significantly Subclonal Events or Significantly Clonal Events of a Tumor Type

The methods described herein can be used to identify regions of the genome that are more likely to contribute to aneuploidy based intratumor heterogeneity in different cancer types. Once identified, these regions can be incorporated into methods described herein that can be used to characterize aneuploidy-based ITH for a patient's tumor without needing to collect multiple samples from the patient.

The methods described herein can be used to characterize portions of the genome that are overrepresented in aneuploidy based intratumor heterogeneity for a tumor type. The portions of the genome that are overrepresented in aneuploidy based intratumor heterogeneity are likely to be aneuploidy in some but not all parts of a tumor of a particular type of tumor. In some instances, the methods can be used to characterize portions of the genome that are overrepresented in having aneuploidy across multiple parts of a tumor of a particular type of tumor. Such chromosome regions can be called significantly subclonal events or significantly clonal events of a particular type of tumor.

The methods depend on collecting samples that can be used as reference samples for a particular type of tumor. A sample can be a reference sample if the sample is one of two or more samples collected from a patient with the particular type of tumor. The patient is the reference patient. In some instances, the methods comprise obtaining reference aneuploidy data for a plurality of tumor samples of the tumor type that can be used as reference tumors for the particular type of tumor. The plurality of reference tumor samples comprises at least two tumor samples obtained at different time points from each reference subject in a plurality of reference subjects; characterizing, for each aneuploidy event in a plurality of aneuploidy events, as unique or shared among the at least two tumor samples from each reference subject in the plurality of reference subjects; determining, for each aneuploidy event in the plurality of aneuploidy events, whether uniqueness of the aneuploidy event within the plurality of aneuploid events is enriched compared to uniqueness of all aneuploidy events within the plurality of aneuploidy events; and characterizing the aneuploidy event as significantly subclonal or significantly clonal based on enrichment of the uniqueness of the aneuploidy event.

The methods described herein can be used to characterize significantly subclonal events or significantly clonal events of a particular tumor type. In some instances, the methods comprise obtaining reference aneuploidy data for a plurality of reference tumor samples of the tumor type, wherein the plurality of reference tumor samples comprises at least two tumor samples obtained at different time points from each reference subject in a plurality of reference subjects; characterizing, for each aneuploidy event in a plurality of aneuploidy events, as unique or shared among the at least two tumor samples from each reference subject in the plurality of reference subjects, determining, for each aneuploidy event in the plurality of aneuploidy events, whether uniqueness of the aneuploidy event within the plurality of aneuploid events is enriched compared to uniqueness of all aneuploidy events within the plurality of aneuploidy events, and characterizing the aneuploidy event as significantly subclonal or significantly clonal based on enrichment of the uniqueness of the aneuploidy event.

In some instances, the methods comprise obtaining reference aneuploidy data for a plurality of reference tumor samples of a tumor type. In some instances, the reference aneuploidy data comprises one or more aneuploidy event annotations. In some instances, the aneuploidy event annotations are characterized as a variation in chromosome number from a base ploidy of the reference sample. The one or more aneuploidy event annotation may be a plurality of aneuploidy events. In some instances, a plurality of aneuploidy events wherein each aneuploidy event is characterized as a gain of a chromosomal portion or a loss of a chromosomal portion. The gain or loss may be relative to the base ploidy of the sample. In some instances, the chromosome portion is a chromosomal arm. in such instances a chromosomal event may be a gain of the chromosomal arm, termed chromosomal arm gain or a loss of the chromosomal arm, termed a chromosomal arm loss. In some instances, the chromosomal portion may be a cytoband. In some instances, the chromosomal portion may range in size from a single base to a full chromosome arm, or any size in between. In some instances, the gain or loss may be binary, such as true for false for a gain or loss of the chromosomal portion. In some instances, the gain or loss may be quantitative such that an event may comprise two gains of a chromosomal portion because the sample has two additional chromosomal portions relative to the base ploidy of the sample.

In some instances, the methods comprise obtaining reference aneuploidy data for a plurality of reference tumor samples of the tumor type. The reference aneuploidy data may comprise one or more aneuploidy event annotations for the plurality of reference tumor samples of the tumor type. In some instances, the one or more aneuploidy event annotations for the reference tumor samples may be the same as the one or more aneuploidy event annotations in the sample aneuploidy data. In some instances, the one or more aneuploidy event annotations for the reference tumor samples may be different from or contain more annotations compared to the one or more aneuploidy event annotations in the sample aneuploidy data.

In some instances, the methods comprise obtaining reference aneuploidy data for a plurality of reference tumor samples of the tumor type, wherein the plurality of reference tumor samples comprises at least two tumor samples obtained at different time points from each reference subject in a plurality of reference subjects. In some instances, the different time points may comprise baseline, less than one year from baseline, between 1 and 3 years from baseline, or greater than 3 years from baseline. In some instances, the time between biopsies is about 50, about 100, about 200, about 300, about 400, about 500, about 600, about 700, about 800, about 900, or more than 1000 days. In some instances, baseline may be when a patient is diagnosed with the tumor or during treatment. In some instances, baseline may be the first tumor sample from a patient. In some instances, the timepoints may be before or after a patient has received any treatment for the cancer.

In some instances, the at least two tumor samples may comprise at least two tumor samples, at least three tumor samples, at least four tumor samples, or at least 5 tumor samples from a reference subject. In some instances, the plurality of reference subjects comprises more patients with the same tumor type or a closely related tumor type. In some instances, the at least two tumor samples, comprise at least two tumor samples from a subject with tumor purity above a threshold percentage. In some instances, the tumor purity is determined using copy number modeling. In some instances, the tumor purity threshold may be greater than 20%, greater than 30%, greater than 40%, greater than 50%, greater than 60%, greater than 70%, greater than 80%, or greater than 90%.

In some instances, the method comprises characterizing, for each aneuploidy event in a plurality of aneuploidy events, as unique or shared among the at least two tumor samples from each reference subject in the plurality of reference subjects. In some instances, for each aneuploidy event in a plurality of aneuploidy events, as unique or shared among the two tumor samples from each reference subject in the plurality of reference subjects. In some instances, an aneuploidy event if shared if it is identified in both of the two tumor samples. In some instances, the aneuploidy event is unique if is identified in one of the two tumor samples.

In some instances, the method comprises characterizing, for each aneuploidy event in a plurality of aneuploidy events, as unique or shared among the at least two tumor samples from each reference subject in the plurality of reference subjects. In some instances, an aneuploidy event is shared if it is identified in more than one of the at least two tumor samples. In some instances, an aneuploidy event may be unique if it is identified in only one of the two or more tumor samples for the reference subject. In some instances, an aneuploidy event may be unique if it is identified in one of three samples from a reference subject.

In some instances, the method comprises characterizing, for each aneuploidy event in a plurality of aneuploidy events, as unique or shared among the at least two tumor samples from each reference subject in the plurality of reference subjects. In some instances, an aneuploidy event is shared if it is identified in all of the at least two tumor samples. In some instances, an aneuploidy event may be unique if it is not identified in all of the two or more tumor samples for the reference subject.

In some instances, the method comprises characterizing, for each aneuploidy event in a plurality of aneuploidy events, as unique or shared among the at least two tumor samples from each reference subject in the plurality of reference subjects. In some instances, characterizing an event as unique or shared among the at least two samples from each reference subject in the plurality of reference subjects comprises characterizing how shared or how unique an aneuploidy event using a level of sharedness or a level of uniqueness. In some instances, characterizing the level of sharedness of an aneuploidy event comprises characterizing the proportion of sample the aneuploidy even is identified in. For example, if two of four tumor samples for a reference subject contain an aneuploidy event, the level of sharing may be 2/4 or 50%. In some instances, characterizing the level of uniqueness of an aneuploidy event comprises characterizing the proportion of sample the aneuploidy even is identified in. For example, if two of four tumor samples for a reference subject contain an aneuploidy event, the level of uniqueness may be 2/4 or 50%.

In some instances, the methods comprise determining, for each aneuploidy event in the plurality of aneuploidy events, whether uniqueness of the aneuploidy event within the plurality of aneuploid events is enriched compared to uniqueness of all aneuploidy events within the plurality of aneuploidy events. In some instances, determining whether uniqueness of the aneuploidy event within the plurality of aneuploid events is enriched comprises determining whether the level of uniqueness of the aneuploidy event.

In some instances, the methods comprise determining, for each aneuploidy event in the plurality of aneuploidy events, whether uniqueness of the aneuploidy event within the plurality of aneuploid events is enriched compared to uniqueness of all aneuploidy events within the plurality of aneuploidy events. In some instances, determining whether uniqueness of an aneuploidy event within the plurality of aneuploid events is enriched compared to uniqueness of all aneuploidy events within the plurality of aneuploidy events comprises comparing how often the aneuploidy event is subclonal, performing a Fisher's exact test, or performing a chi-squared test. In some instances determining whether uniqueness of an aneuploidy event within the plurality of aneuploid events is enriched compared to uniqueness of all aneuploidy events within the plurality of aneuploidy events comprises filing out a two by two contingency tables wherein the top left value represents how often a particular aneuploidy event is shared among two samples from a reference subject in the plurality of reference subjects, the top right value represents how often a particular aneuploidy event is unique among two samples from a reference subject in the plurality of reference subjects, the bottom left value represents how often all other identified aneuploidy events are shared among two samples from a reference subject in the plurality of reference subjects, and the bottom right value represents how often all other identified aneuploidy events are unique among two samples from a reference subject in the plurality of reference subjects. In some instance, the methods comprise an enrichment test for each of the plurality of aneuploidy events.

In some instances, the methods comprise characterizing the aneuploidy event as significantly subclonal or significantly clonal based on enrichment of the uniqueness of the aneuploidy event. In some instances, an enrichment test comprises generating an odds ratio that characterizes the enrichment of the uniqueness of the aneuploidy event. In some instances, the odds ratio or fold change in odds ratio is used to classify an aneuploidy event as significantly clonal or significantly subclonal. In some instances, events are significantly clonal or significantly subclonal if the change in odds ratio is beyond a threshold. In some instances, an aneuploidy event is significantly subclonal if the fold change in odds ratio is beyond a cutoff in the negative direction, such as more negative than about −1.5. In some instances, an aneuploidy event is significantly clonal if the fold change in odds ratio beyond a cutoff in the positive direction, such as greater than about 1.5. In some instances, the fold change in odds ratio is log 2 of the odds ratio. In some instances, an aneuploidy event is significantly subclonal if log 2 (odds ratio) is beyond a threshold in the negative direction. In some instances, an aneuploidy event is significantly clonal if log 2 (odds ratio) is beyond a threshold in the positive direction.

In some instances, subclonal refers to an aneuploidy event that is more likely to be unique to one of two or more samples from a cancer patient. In some instances, a subclonal aneuploidy event is significantly subclonal when the results of an enrichment test provide high confidence that an aneuploidy event is likely to unique to one of two or more samples from a cancer patient with a certain tumor type. In some instances, subclonal may refer to an aneuploidy event that occurs in a subset of cancer cells in the tumor for a certain tumor type.

In some instances, clonal refers to an aneuploidy event that is more likely to be shared among two or more samples from a cancer patient. In some instances, a clonal aneuploidy event is significantly clonal when the results of an enrichment test provide high confidence that an aneuploidy event is likely to be shared among two or more samples from a cancer patient with a certain tumor type. In some instances, clonal may refer to an aneuploidy event that occurs in all cancer cells in the tumor for a certain tumor type.

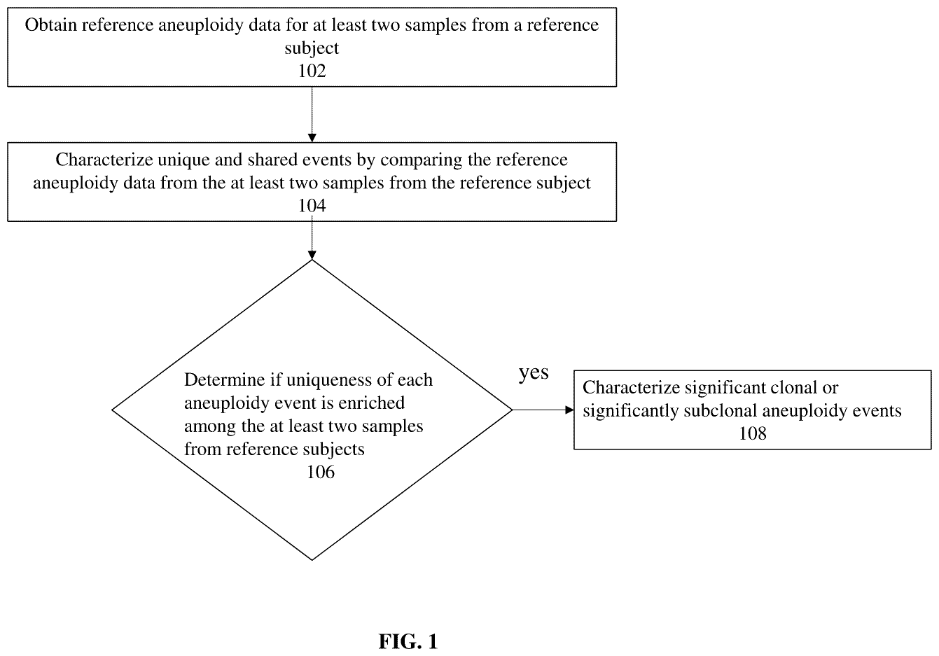

FIG. 1 provides a non-limiting example of a method to characterize significantly subclonal or significantly subclonal events of a tumor type. Reference aneuploidy data from a plurality of reference tumor samples of the tumor type are obtained in 102. The plurality of reference tumor samples comprises at least two tumor samples that were collected at different time points for each of the reference subjects in the plurality of references subjects. The reference aneuploidy data is then used in 104 to characterized unique and shared aneuploidy events. Aneuploidy events are characterized as shared or unique using the matched reference tumor samples from each of the reference subjects. These data are then used in 106, wherein the uniqueness of each of the aneuploidy events in the plurality of aneuploidy events is determined by testing for enrichment of the uniqueness of each event compared to the uniqueness of all aneuploidy events. If the uniqueness of the aneuploidy is enriched the event is characterized in 108 as significantly subclonal or significantly clonal events. An aneuploidy event may be significantly subclonal if the fold change in the odds ratio determined by the enrichment analysis in 206 is beyond a cutoff in the negative direction. An aneuploidy event may be significantly if the fold change in the odds ratio determined by the enrichment analysis in 206 is beyond a cutoff in the positive direction.

Methods for Characterizing Aneuploidy Based Intratumor Heterogeneity for a Tumor of a Tumor Type from a subject

The disclosed methods can be used to characterize the variation in aneuploidy across different cells of a patient's tumor without the need to collect multiple samples from a tumor or test for variation at individual cell levels. Using aneuploidy events detected in one tumor sample and the type of cancer, the methods can be used to characterize aneuploidy based intratumor heterogeneity (ITH) for the patient. The aneuploidy-based ITH characterization for the tumor may be generated by analyzing a tumor sample collected from the patient.

The disclosed methods can be used to characterize aneuploidy based intratumor heterogeneity (ITH) for a tumor type from a subject. The disclosed methods comprise, obtaining sample aneuploidy data for the tumor; calling subclonal aneuploidy events in the sample aneuploidy data, wherein a subject aneuploidy event is characterized as subclonal based on a comparison of the subject aneuploidy event to a corresponding reference aneuploidy event for the tumor type, wherein the reference aneuploidy event had been characterized by: obtaining reference aneuploidy data for a plurality of reference tumor samples of the tumor type; wherein the plurality of reference tumor samples comprises at least two tumor samples obtained at different time points from each reference subject in a plurality of reference subjects; characterizing, for each aneuploidy event in a plurality of aneuploidy events, as unique or shared among the at least two tumor samples from each reference subject in the plurality of reference subjects; determining, for each aneuploidy event in the plurality of aneuploidy events; whether uniqueness of the aneuploidy event within the plurality of aneuploid events is enriched compared to uniqueness of all aneuploidy events within the plurality of aneuploidy events; and characterizing the aneuploidy event as significantly subclonal or significantly clonal based on enrichment of the uniqueness of the aneuploidy event; and generating an intratumor heterogeneity score for the tumor sample based on a number of called significantly subclonal events in the sample aneuploidy data for the tumor sample.

In some instances, the methods comprise obtaining sample aneuploidy data for the tumor. In some instances, the sample aneuploidy data for the tumor can be generated from a single sample taken from the tumor. In some instances, the single sample taken from the tumor may be from a liquid biopsy or a tissue biopsy. In some instances, the reference sample data comprises one or more aneuploidy event annotations. In some instances, the aneuploidy event annotations are characterized as a variation in chromosome number from a base ploidy of the sample. The one or more aneuploidy event annotation may be a plurality of aneuploidy events. In some instances, a plurality of aneuploidy events wherein each aneuploidy event is characterized as a gain of a chromosomal portion or a loss of a chromosomal portion. The gain or loss may be relative to the base ploidy of the sample. In some instances, the chromosome portion is a chromosomal arm. In such instances a chromosomal event may be a gain of the chromosomal arm, termed chromosomal arm gain or a loss of the chromosomal arm, termed a chromosomal arm loss. In some instances, the chromosomal portion may be a cytoband. In some instances, the chromosomal portion may range in size from a single base to a full chromosome arm, or any size in between. In some instances, the gain or loss may be binary, such as true for false for a gain or loss of the chromosomal portion. In some instances, the gain or loss may be quantitative. The quantification may be the number of copies of the chromosomal portion compared to the base anaploidy of the sample. In a non limiting example, there may a quantitative gain of 2 when the copy number of the region is 4 because the sample ploidy is 2. In some aspects, the gain or loss may represent a deviation from a copy number of 2.

In some instances, the methods comprise calling subclonal aneuploidy events in the sample aneuploidy data, wherein a subject aneuploidy event is characterized as subclonal based on a comparison of the subject aneuploidy event to a corresponding reference aneuploidy event for the tumor type. In some instances, the reference aneuploidy event had been characterized according to methods described herein. In some instances, the methods comprise obtaining reference aneuploidy data for a plurality of reference tumor samples of the tumor type, wherein the plurality of reference tumor samples comprises at least two tumor samples obtained at different time points from each reference subject in a plurality of reference subjects, characterizing, for each aneuploidy event in a plurality of aneuploidy events, as unique or shared among the at least two tumor samples from each reference subject in the plurality of reference subjects, determining, for each aneuploidy event in the plurality of aneuploidy events, whether uniqueness of the aneuploidy event within the plurality of aneuploid events is enriched compared to uniqueness of all aneuploidy events within the plurality of aneuploidy events, and characterizing the aneuploidy event as significantly subclonal significantly based on enrichment of the uniqueness of the aneuploidy event.

In some instances, the methods comprise calling clonal aneuploidy events in the sample aneuploidy data, wherein a subject aneuploidy event is characterized as subclonal based on a comparison of the subject aneuploidy event to a corresponding reference aneuploidy event for the tumor type. In some instances, the reference aneuploidy event had been characterized according to methods described herein. In some instances, the methods comprise obtaining reference aneuploidy data for a plurality of reference tumor samples of the tumor type, wherein the plurality of reference tumor samples comprises at least two tumor samples obtained at different time points from each reference subject in a plurality of reference subjects, characterizing, for each aneuploidy event in a plurality of aneuploidy events, as unique or shared among the at least two tumor samples from each reference subject in the plurality of reference subjects, determining, for each aneuploidy event in the plurality of aneuploidy events, whether uniqueness of the aneuploidy event within the plurality of aneuploid events is enriched compared to uniqueness of all aneuploidy events within the plurality of aneuploidy events, and characterizing the aneuploidy event as significantly clonal significantly based on enrichment of the uniqueness of the aneuploidy event.

In some instances, calling subclonal aneuploidy events in the sample aneuploidy data comprises comparing the list of sample aneuploidy events to significantly clonal and significantly subclonal aneuploidy events identified for the tumor type.

In some instances, the methods comprise generating an intratumor heterogeneity score for the tumor sample based on a number of called significantly subclonal events or significantly clonal in the sample aneuploidy data for the tumor sample. In some instances, the ITH score is a sum of the aneuploidy events from the sample aneuploidy data that are represented in the significantly subclonal events for the tumor type.

In some instances, the methods comprise generating an intratumor heterogeneity score for the tumor sample based on a number of called significantly clonal events in the sample aneuploidy data for the tumor sample.

In some instances, the methods comprise, generating an intratumor heterogeneity indicator, wherein the intratumor heterogeneity indicator relates to the relationship between the intratumor heterogeneity score and a determined threshold. In some instances, the determined threshold is a predetermined threshold. In some instances, the threshold is predetermined using the reference sample data. In some instances, the determined threshold comprises an upper threshold and a lower threshold. In some instances, the determined threshold is determined using the interquartile range for the number of significantly subclonal events in the reference samples from the plurality or reference patients with the tumor type. In some instances, the intratumor heterogeneity indicator is high if the intratumor heterogeneity metric is greater than or equal to the upper threshold, the intratumor heterogeneity indicator is intermediate if the intratumor heterogeneity metric is greater than the lower threshold and less than the lower threshold, and the intratumor heterogeneity indicator is low if intratumor heterogeneity metric is less than or equal to the lower threshold. In some instances, the high intratumor heterogeneity score relates to poor prognosis, quick resistance to cancer therapies, or poor outcomes. In some instances, the methods comprise generating an aneuploidy burden score by integrating the intratumor heterogeneity score with digital pathology-based heterogeneity, single cell heterogeneity scores, radiological heterogeneity scores, aneuploidy burden, cytoband features, CN segment features for the tumor. In some instances, digital pathology-based heterogeneity comprises the results of identifying clonal and subclonal aneuploidy events in pathology images of a tumor. In some instances, single cell heterogeneity scores comprise the results of performing single cell sequencing on tumor cells and using computational methods to identify clonal and subclonal aneuploidy events. In some instances, radiological heterogeneity scores comprise the results of identifying clonal and subclonal aneuploidy events by medical imaging of a tumor. In some instances, aneuploidy burden may refer to an amount of aneuploidy events in a sample. In some instances, cytoband features may refer to variation at a cytoband across tumor cells. In some instances, CN segment features copy number variation across tumor cells. In some instances, ITH scores generated using various levels of chromosome portions, such as full chromosome, chromosome arm, and cytoband can be integrated. In some instances, the methods comprise generating an aneuploidy burden score by integrating the intratumor heterogeneity score with other scores known in the art measuring aneuploidy-based ITH or other aspects of intratumor heterogeneity. In some instances, the methods comprise generating an aneuploidy burden score by integrating the intratumor heterogeneity score with an outcome of a machine learning model known in the art. In some instances, the machine learning model may be trained to generate an aneuploidy burden score based on one or more of the ITH metrics described herein.

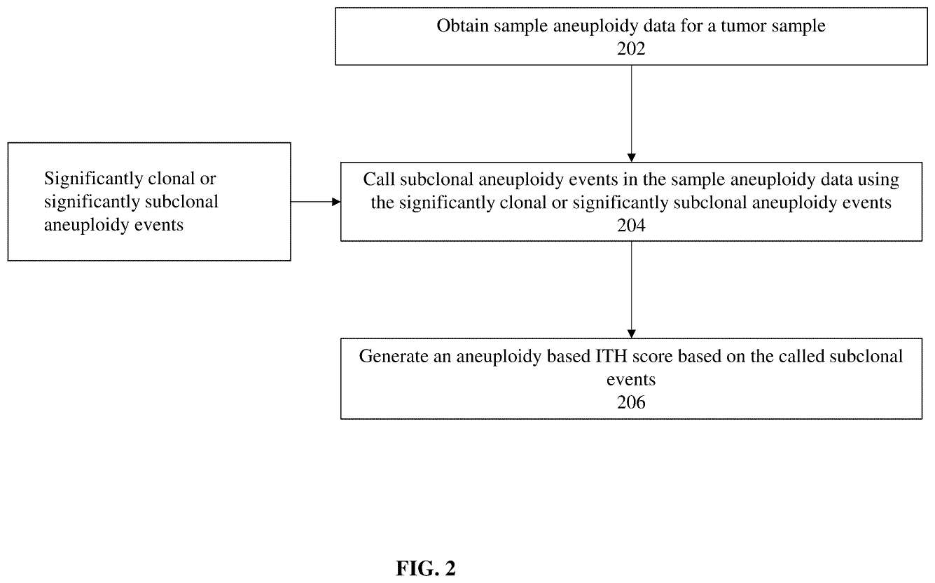

FIG. 2 provides a non-limiting example of a method of characterizing aneuploidy based intratumor heterogeneity for a tumor of a tumor type from a subject. Sample aneuploidy data for the tumor is obtained in 202. The sample aneuploidy data for a tumor sample comprises aneuploidy events identified in a single sample of the sample tumor. The sample aneuploidy data and significantly clonal or significantly subclonal aneuploidy events generated using the method of FIG. 1 can be used as inputs for 204, wherein subclonal aneuploidy events for the sample are called. The method in 204 may comprise matching the aneuploidy events in the sample aneuploidy data that are also significantly subclonal aneuploidy events for the same tumor type. The called aneuploidy events from 204 can be input to 206 wherein an aneuploidy-based ITH score is generated for the tumor sample. The method in 206 may comprise counting the number of called subclonal events identified in 204. The method disclosed in FIG. 2 can use used to generate an aneuploidy based ITH score for a tumor sample of any cancer type by modifying the significantly clonal or significantly subclonal aneuploidy events that are used as input to 204.

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) performing a methylation conversion reaction to convert, e.g., non-methylated cytosine to uracil, (v) amplifying the nucleic acid molecules (e.g., using a polymerase chain reaction (PCR) amplification technique, a non-PCR amplification technique, or an isothermal amplification technique), (vi) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vii) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, (viii) combining the nucleic acid sequence data (including, e.g., variant data, copy number data, methylation status data, etc., of the sequenced nucleic acid molecules) with other biomarker data modalities including, but not limited to, proteomics-based biomarker data (e.g., the detection of specific polypeptides, such as proteins) or fragmentomics-based biomarker data (e.g., the detection of certain attributes related to nucleic acid fragments, such as fragment size or the sequences of fragment ends), to determine, for example, the presence of ctDNA in the sample and/or to determine a diagnostic, prognostic, and/or treatment response prediction for the subject, and (ix) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA). In some instances, the cell-free DNA (cfDNA), or a portion thereof, may comprise circulating tumor DNA (ctDNA). In some instances, the liquid biopsy sample may comprise a combination of cell-free DNA (cfDNA) and circulating tumor DNA (ctDNA).

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods for characterizing aneuploidy based intratumor heterogeneity may be used to diagnose (or as part of a diagnosis of) the presence of disease or other condition (e.g., cancer) in a subject. In some instances, the disclosed methods may be applicable to diagnosis of any of a variety of cancers as described elsewhere herein.

In some instances, the disclosed methods for characterizing aneuploidy based intratumor heterogeneity may be used to select a subject (e.g., a patient) for a clinical trial based on the aneuploidy based intratumor heterogeneity value. In some instances, patient selection for clinical trials based on, e.g., identification of a high intratumor heterogeneity score, may accelerate the development of targeted therapies and improve the healthcare outcomes for treatment decisions.

### Methods for Determining an Intratumor Heterogeneity Score for a NSCLC Tumor

Disclosed herein are methods that can be used to determine and use an intratumor heterogeneity score for a NSCLC tumor. In some instances, the methods comprise, obtaining sample aneuploidy data for a non-small cell lung cancer (NSCLC) tumor of a subject; calling subclonal aneuploidy events in the sample aneuploidy data, wherein a subject aneuploidy event is characterized as subclonal based on a comparison of the subject aneuploidy event to corresponding NSCLC reference aneuploidy events included in the NSCLC significantly subclonal events; and generating an intratumor heterogeneity score based on a number of aneuploidy events in the aneuploidy event data included in the NSCLC significantly subclonal events.

In some instances, the list of NSCLC significantly clonal events comprise a plurality of NSCLC reference aneuploidy events. In some instances, the NSCLC reference aneuploidy events comprise arm level chromosome gains of 2p, 2q, 3p, 4q, 6q, 10q, 12q, 13q, 15q, 16q, 17p, 18q, 19p, 21q, and 22q, and arm level chromosomal losses of 1q, 2p, 2q, 3q, 5p, 6p, 7p, 7q, 11p, 11q, 12q, 16p, 17q, and 20q. In some instances, the NSCLC reference aneuploidy events may be the significantly subclonal aneuploidy events that can be used as input into 204 of FIG. 2 in order to generate an ITH score for a tumor sample obtained from a patient with NSCLC.

In some instances, the methods described herein are used to obtain sample aneuploidy data from a sample taken from a NSCLC tumor. In some instances, the sample is taken from a single tissue biopsy of a NSCLC tumor or from a single liquid biopsy from a patient with NSCLC. In some instances, the sample aneuploidy data comprises one or more aneuploidy annotations. In some instances, the one or more aneuploidy event annotations are characterized as a variation in chromosome number from a base ploidy of the sample. In some instances, the one or more aneuploidy event annotations comprise a plurality of aneuploidy events. In some instances, an aneuploidy event in the plurality of aneuploidy events is an arm level chromosome gain or an arm level chromosomal loss.

In some instances, generating the intratumor heterogeneity score comprises summing the number of called significantly subclonal events in the sample aneuploidy event data for the tumor sample. In some instances, the methods further comprise generating an intratumor heterogeneity indicator, wherein the intratumor heterogeneity indicator relates to the relationship between the intratumor heterogeneity score and a determined threshold. In some instances, a determined threshold comprises an upper threshold and a lower threshold. In some instances, the intratumor heterogeneity indicator is high if the intratumor heterogeneity metric is greater than or equal to the upper threshold, the intratumor heterogeneity indicator is intermediate if the intratumor heterogeneity metric is greater than the lower threshold and less than the lower threshold, and the intratumor heterogeneity indicator is low if intratumor heterogeneity metric is less than or equal to the lower threshold. In some instances, the upper threshold is 2, 3, 4, 5, or 6. In some instances the lower threshold is 0, 1, or 2. In some instances, the upper threshold is 4 and the lower threshold is 1. In some instances, a high intratumor heterogeneity score relates to poor prognosis, quick resistance to cancer therapies as described herein, or poor outcomes.

### Methods for Determining an Intratumor Heterogeneity Score for an Ovarian Cancer Tumor

Disclosed herein are methods that can be used to determine and use an intratumor heterogeneity score for an ovarian tumor. In some instances, the methods comprise, obtaining sample aneuploidy data for an ovarian tumor of a subject; calling subclonal aneuploidy events in the sample aneuploidy data, wherein a subject aneuploidy event is characterized as subclonal based on a comparison of the subject aneuploidy event to corresponding ovarian reference aneuploidy events included in the ovarian significantly subclonal events; and generating an intratumor heterogeneity score based on a number of aneuploidy events in the aneuploidy event data in the ovarian cancer significantly subclonal events.

In some instances, the list of ovarian significantly clonal events comprises a plurality of ovarian reference aneuploidy events. In some instances, the plurality of ovarian reference aneuploidy events comprise arm level chromosome gains of 1p, 3p, 4q, 11p, 12q, 13q, 16p, 16q, 17q, 19q, 21q and 22q, and arm level chromosomal losses of 1q, 2p, 2q, 3p, 5p, 6p, 7q, 8q, 10p, 12q, 17q, 20p, 20q, and 21q. In some instances, the ovarian reference aneuploidy events may be the significantly subclonal aneuploidy events that are used as input into 204 of FIG. 2 in order to generate an ITH score for a tumor sample obtained from a patient with ovarian cancer.

In some instances, the methods described herein are used to obtain sample aneuploidy data from a sample taken from an ovarian tumor. In some instances, the sample is taken from a single tissue biopsy of an ovarian tumor or from a single liquid biopsy from a patient with ovarian cancer. In some instances, the sample aneuploidy data comprises one or more aneuploidy annotations. In some instances, the one or more aneuploidy event annotations are characterized as a variation in chromosome number from a base ploidy of the sample. In some instances, the one or more aneuploidy event annotations comprise a plurality of aneuploidy events. In some instances, an aneuploidy event in the plurality of aneuploidy events is an arm level chromosome gain or an arm level chromosomal loss.

In some instances, generating the intratumor heterogeneity score comprises summing the number of called significantly subclonal events in the sample aneuploidy event data for the tumor sample. In some instances, the methods further comprise generating an intratumor heterogeneity indicator, wherein the intratumor heterogeneity indicator relates to the relationship between the intratumor heterogeneity score and a determined threshold. In some instances, a determined threshold comprises an upper threshold and a lower threshold. In some instances, the intratumor heterogeneity indicator is high if the intratumor heterogeneity metric is greater than or equal to the upper threshold, the intratumor heterogeneity indicator is intermediate if the intratumor heterogeneity metric is greater than the lower threshold and less than the lower threshold, and the intratumor heterogeneity indicator is low if intratumor heterogeneity metric is less than or equal to the lower threshold. In some instances, the upper threshold is 2, 3, 4, 5, or 6. In some instances the lower threshold is 0, 1, or 2. In some instances, the upper threshold is 4 and the lower threshold is 1. In some instances, a high intratumor heterogeneity score relates to poor prognosis, quick resistance to cancer therapies as described herein, or poor outcomes.

### Methods for Determining an Intratumor Heterogeneity Score for a Breast Cancer Tumor

Disclosed herein are methods that can be used to determine and use an intratumor heterogeneity score for a breast tumor. In some instances, the methods comprise, obtaining sample aneuploidy data for a breast tumor of a subject; calling subclonal aneuploidy events in the sample aneuploidy data, wherein a subject aneuploidy event is characterized as subclonal based on a comparison of the subject aneuploidy event to corresponding breast cancer reference aneuploidy events included in the breast cancer significantly subclonal events; and generating an intratumor heterogeneity score based on a number of aneuploidy events in the aneuploidy event data in the breast cancer significantly subclonal events.

In some instances, the list of breast cancer significantly clonal events comprises a plurality of breast cancer reference aneuploidy events. In some instances, the plurality of breast cancer reference aneuploidy events comprise arm level chromosome gains of 1p, 2p, 2q, 3p, 4p, 4q, 9q, 10q, 11p, 11q, 13q, 14q, 15q, 16q, 17p, 18p, 18q, 19p, 19q, 21q and 22q and arm level chromosomal losses of 1p, 2p, 2q, 3q, 5p, 6p, 7p, 8q, 10p, 16p, 19p, 19q, 20p, and 20q. In some instances, the breast cancer reference aneuploidy events may be the significantly subclonal aneuploidy events that are used as input into 204 of FIG. 2 in order to generate an ITH score for a tumor sample obtained from a patient with breast cancer.

In some instances, the methods described herein are used to obtain sample aneuploidy data from a sample taken from a breast tumor. In some instances, the sample is taken from a single tissue biopsy of a breast tumor or from a single liquid biopsy from a patient with breast cancer. In some instances, the sample aneuploidy data comprises one or more aneuploidy annotations. In some instances, the one or more aneuploidy event annotations are characterized as a variation in chromosome number from a base ploidy of the sample. In some instances, the one or more aneuploidy event annotations comprise a plurality of aneuploidy events. In some instances, an aneuploidy event in the plurality of aneuploidy events is an arm level chromosome gain or an arm level chromosomal loss.

In some instances, generating the intratumor heterogeneity score comprises summing the number of called significantly subclonal events in the sample aneuploidy event data for the tumor sample. In some instances, the methods further comprise generating an intratumor heterogeneity indicator, wherein the intratumor heterogeneity indicator relates to the relationship between the intratumor heterogeneity score and a determined threshold. In some instances, a determined threshold comprises an upper threshold and a lower threshold. In some instances, the intratumor heterogeneity indicator is high if the intratumor heterogeneity metric is greater than or equal to the upper threshold, the intratumor heterogeneity indicator is intermediate if the intratumor heterogeneity metric is greater than the lower threshold and less than the lower threshold, and the intratumor heterogeneity indicator is low if intratumor heterogeneity metric is less than or equal to the lower threshold. In some instances, the upper threshold is 1, 2, 3, 4, or 5. In some instances the lower threshold is 0, or 1. In some instances, the upper threshold is 5 and the lower threshold is 1. In some instances, a high intratumor heterogeneity score relates to poor prognosis, quick resistance to cancer therapies as described herein, or poor outcomes.

In some instances, a high intratumor heterogeneity score relates to poor outcomes, such as short survival. In some instances, a high intratumor heterogeneity score relates to a shorter progression free survival for the individual than an individual with an intermediate or low intratumor heterogeneity score. In some instances, a subject with a high intratumor heterogeneity is projected to have a progression free survival of about 11 months. In some instances, a subject with an intermediate intratumor heterogeneity is projected to have a progression free survival of about 15 months. In some instances, a subject with a low intratumor heterogeneity is projected to have a progression free survival of about 17 months. In some instances, progression free survival is progression free survival when the subject receives CDK4/6 inhibitor and endocrine therapy.

In some instances, breast tumor sample has been collected from a subject diagnosed with breast cancer. In some instances, the breast cancer was stage 1 at diagnosis. In some instances, the breast cancer is HR+ HER2− advanced breast cancer. In some instances, the breast cancer was stage 2 at diagnosis. In some instances, the CDK4/6 inhibitor and endocrine therapy are given as first line, second line, or late line therapy.

### Methods for Selecting Cancer Treatment Using Aneuploidy Based Intratumor Heterogeneity

In some instances, the disclosed methods for characterizing aneuploidy based intratumor heterogeneity may be used to select an appropriate therapy or treatment (e.g., an anti-cancer therapy or anti-cancer treatment) for a subject. In some instances, for example, the anti-cancer therapy or treatment may comprise use of a poly(ADP-ribose) polymerase inhibitor (PARPi), a platinum compound, chemotherapy, radiation therapy, a targeted therapy, an immunotherapy, a neoantigen-based therapy, surgery, or any combination thereof. In some instances, for example, the anti-cancer therapy or treatment may comprise an antibody-drug conjugate (ADC).

In some instances, the anti-cancer therapy or treatment may comprise a targeted anti-cancer therapy or treatment (e.g., a monoclonal antibody-based therapy, an enzyme inhibitor-based therapy, an antibody-drug conjugate therapy, a hormone therapy, and/or a targeted radiotherapy) that targets specific molecules required for cancer cell growth, division, and spreading In some instances, the targeted anti-cancer therapy or treatment may comprise abemaciclib (Verzenio), abiraterone acetate (Zytiga), acalabrutinib (Calquence), ado-trastuzumab emtansine (Kadcyla), afatinib dimaleate (Gilotrif), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some instances, the anti-cancer therapy or treatment may comprise an immunotherapy (e.g., a cancer treatment that acts by stimulating the immune system to fight cancer). In some instances, the immunotherapy can be, for example, an immune system modulator (e.g., a cytokine, such as an interferon or interleukin), an immune checkpoint inhibitor (such as an anti-PD-1 or anti-PD-L1 antibody), a T-cell transfer therapy (e.g., a tumor infiltrating lymphocyte (TIL) therapy in lymphocytes extracted from a patient's tumor are selected for their ability to recognize tumor cells and propagated prior to reintroduction into the patient, or a CAR T-cell therapy in which a patient's T-cells are modified to express the CAR protein prior to reintroduction into the patient), a monoclonal antibody-based therapy (e.g., a monoclonal antibody that binds to cell surface markers on cancer cells to facilitate recognition by the immune system), or a cancer treatment vaccine (e.g., a vaccine based on tumor cells, tumor-associated neoantigens, or dendritic cells, etc., that stimulates the immune system to fight cancer).

In some instances, the anti-cancer therapy or treatment may comprise a neoantigen-based therapy. Non-limiting examples of neoantigen-based therapies include T-cell receptor (TCR) engineered T-cell (TCR-T) therapies, chimeric antigen receptor T-cell (CAR-T) therapies, TCR bispecific antibody therapies, and cancer vaccines. TCR-T therapies are produced by genetically engineering a patient's T-cells to express T-cell receptors that are specific to neoantigens of interest, and then infusing them back into the patient. CAR-T therapies are produced by genetically engineering a patient's T-cells to express chimeric antigen receptor molecules which contain an intracellular signaling and co-signaling domain as well as an extracellular antigen-binding domain; CAR-T therapies don't always rely on neoantigen presentation, but can be designed to be directed towards neoantigens. TCR bispecific antibody therapies are small, engineered antibody molecules that comprise a neoantigen-specific TCR on one end and a CD3-directed single-chain variable fragment on the other end. Cancer vaccines can include RNA molecules, DNA molecules, peptides, or a combination thereof that are designed to boost the immune system's ability to find and destroy neoantigen-presenting cells.

In some instances, the hormone therapy is an endocrine therapy. Endocrine therapies may be used to treat hormone-receptor positive cancers, such as hormone-receptor positive breast cancer. The endocrine therapy may comprise a selective estrogen receptor modulator (SERMs), an aromatase inhibitor (Ais) or an estrogen receptor downregulatory (ERD). The endocrine therapy may comprise a selective estrogen receptor degrader (SERD).

In some embodiments, the anti-cancer therapy comprises a CDK4/6 inhibitor. In some embodiments, the methods provided herein comprise administering to the individual a CDK4/6 inhibitor, e.g., in combination with another anti-cancer therapy. In some embodiments, the CDK4/6 inhibitor is ribociclib (KISQALI®, LEE011), palbociclib (PD0332991, IBRANCE®), or abemaciclib (LY2835219). In some instances, the disclosed methods for characterizing aneuploidy based intratumor heterogeneity may be used in treating a disease (e.g., a cancer) in a subject. For example, in response to determining aneuploidy based intratumor heterogeneity using any of the methods disclosed herein, an effective amount of an anti-cancer therapy or anti-cancer treatment may be administered to the subject.

In some instances, the disclosed methods for characterizing aneuploidy based intratumor heterogeneity may be used for monitoring disease progression or recurrence (e.g., cancer or tumor progression or recurrence) in a subject. For example, in some instances, the methods may be used to determine aneuploidy based intratumor heterogeneity in a first sample obtained from the subject at a first time point, and used to determine aneuploidy based intratumor heterogeneity in a second sample obtained from the subject at a second time point, where comparison of the first determination of aneuploidy based intratumor heterogeneity and the second determination of aneuploidy based intratumor heterogeneity allows one to monitor disease progression or recurrence. In some instances, the first time point is chosen before the subject has been administered a therapy or treatment, and the second time point is chosen after the subject has been administered the therapy or treatment.

In some instances, the disclosed methods may be used for adjusting a therapy or treatment (e.g., an anti-cancer treatment or anti-cancer therapy) for a subject, e.g., by adjusting a treatment dose and/or selecting a different treatment in response to a change in the determination of an aneuploidy based intratumor heterogeneity metric.

In some instances, the value of aneuploidy based intratumor heterogeneity determined using the disclosed methods may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods for characterizing aneuploidy based intratumor heterogeneity may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), which is a next-generation sequencing (NGS) approach used to assess hundreds of genes (including relevant cancer biomarkers) in a single assay. Inclusion of the disclosed methods for characterizing aneuploidy based intratumor heterogeneity as part of a genomic profiling process (or inclusion of the output from the disclosed methods for characterizing aneuploidy based intratumor heterogeneity as part of the genomic profile of the subject) can improve the validity of, e.g., disease detection calls and treatment decisions, made on the basis of the genomic profile by, for example, independently confirming the presence of low, high, or intermediate intratumor heterogeneity in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

### Methods of Treatment Using Aneuploidy Based Intratumor Heterogeneity

In some instances, the disclosed methods for characterizing aneuploidy based intratumor based heterogeneity may be used in methods of treating or delaying progression of a cancer in an individual. In some instances, the cancer may by NSCLC, ovarian cancer or breast cancer. In some instances, the methods comprise acquiring knowledge of aneuploidy based intratumor based heterogeneity or an intratumor heterogeneity indicator in a sample from the individual and administering a treatment based on said knowledge. The aneuploidy based intratumor based heterogeneity may provide a basis for the effective amount of the treatment administered or the composition of the treatment administered. The treatment may a hormone therapy such as an endocrine therapy. In some instances, the treatment may be a CDK4/6 inhibitor.

In some embodiments, the methods of treating or delaying progression of a NSCLC of the disclosure in an individual, comprise administering to the individual a therapeutically effective amount of an anti-cancer therapy. In some embodiments, the anti-cancer therapy comprises chemotherapeutic agents, immunotherapies targeting EGFR, NTRK, ALK, ROS1, or RET.

In some embodiments, the methods of treating or delaying progression of a ovarian cancer of the disclosure in an individual, comprise administering to the individual a therapeutically effective amount of an anti-cancer therapy. In some embodiments, the anti-cancer therapy comprises a PARP inhibitor (PARPi) and/or a platinum based chemotherapeutic agent.

In some embodiments, the methods of treating or delaying progression of a breast cancer of the disclosure in an individual, comprise administering to the individual a therapeutically effective amount of an anti-cancer therapy, such as endocrine therapy or CDK4/6 inhibitor. In some embodiments, the sample is a tumor biopsy sample or a liquid biopsy sample. In some embodiments, the sample comprises cells from the cancer or is obtained from cells from the cancer. The methods of treatment disclosed herein may include any of the anti-cancer therapies and/or therapeutic agents, such as an endocrine therapy or a CDK4/6 inhibitor.

In some aspects, the methods of treating or delaying progression of a cancer, comprise administering to the individual a combination therapy when a sample from the cancer has a high intratumor based heterogeneity indicator. Combination treatments may be used for treating highly heterogenous tumors because the treatment interacts with multiple targets.

In some aspects, the methods of treating or delaying progression of a cancer, comprise administering to the individual an immunotherapy therapy when a sample from the cancer has a high intratumor based heterogeneity indicator. Immunotherapies may be effective for treating highly heterogenous tumors.

In some aspects, the methods of treating or delaying progression of a cancer, comprise administering to the individual a combination therapy and an immunotherapy therapy when a sample from the cancer has a high intratumor based heterogeneity indicator. Immunotherapies and combination therapies may be effective for treating highly heterogenous tumors.

Methods if Diagnosing, Assessing, Screening, Monitoring or Predicting Survival Using Aneuploidy based intratumor heterogeneity

In some instances, the disclosed methods for characterizing aneuploidy based intratumor based heterogeneity may be used in methods of diagnosing or assessing a cancer in an individual. In some instances, the cancer may be NSCLC, ovarian cancer, or breast cancer. In some instances, the methods comprise acquiring knowledge of aneuploidy based intratumor based heterogeneity or an intratumor heterogeneity indicator in a sample from the individual and diagnosing or assessing the cancer based on said knowledge. In some embodiments, the diagnosis or assessment identifies the cancer as likely to respond to an anti-cancer therapy, e.g., an anti-cancer therapy. In some instances, the anti-cancer therapy is an endocrine therapy or a CDK4/6 inhibitor. In some embodiments, a low intratumor heterogeneity indicator of the sample identifies the cancer as likely to respond to an anti-cancer therapy, such as an endocrine therapy or a CDK4/6 inhibitor. In some embodiments, an intermediate intratumor heterogeneity indicator of the sample identifies the cancer as likely to respond to an anti-cancer therapy such as an endocrine therapy or a CDK4/6 inhibitor. In some embodiments, a high intratumor heterogeneity indicator of the sample identifies the cancer as less likely to respond to an anti-cancer therapy such as an endocrine therapy or a CDK4/6 inhibitor. For example, a cancer with a sample identified by a high intratumor heterogeneity indicator may be less likely to respond to the anti-cancer therapy than a cancer with a sample identified as intermediate or low intratumor heterogeneity indicator. In some embodiments, the sample is tissue biopsy sample or liquid biopsy sample. In some embodiments, the sample comprises cells from the cancer or is obtained from cells from the cancer. In some embodiments, the individual has a cancer, is suspected of having a cancer, is being tested for a cancer, is being treated for a cancer, or is being tested for a susceptibility to a cancer.

In some instances, the disclosed methods for characterizing aneuploidy based intratumor based heterogeneity may be used in methods of monitoring a cancer in an individual. In some instances, the cancer may by NSCLC, ovarian cancer or breast cancer. In some instances, the methods comprise acquiring knowledge of aneuploidy based intratumor based heterogeneity or an intratumor heterogeneity indicator in a sample from the individual and diagnosing or assessing the cancer based on said knowledge. In some instances, monitoring may comprise characterizing aneuploidy based intratumor based heterogeneity in two or more samples from the individual. In some instances, a change in aneuploidy based intratumor based heterogeneity, such as but not limited to intratumor heterogeneity indicators changing from low to intermediate or to high may indicate cancer progression. In some embodiments, the two or more samples are tissue biopsy samples and/or liquid biopsy samples. In some embodiments, the sample comprises cells from the cancer or is obtained from cells from the cancer. In some embodiments, the individual has a cancer, is suspected of having a cancer, is being tested for a cancer, is being treated for a cancer, or is being tested for a susceptibility to a cancer.

In some instances, the disclosed methods for characterizing aneuploidy based intratumor based heterogeneity may be used in methods of predicting survival of an individual having a cancer. In some instances, the cancer may by NSCLC, ovarian cancer or breast cancer. In some instances, the methods comprise acquiring knowledge of aneuploidy based intratumor based heterogeneity or an intratumor heterogeneity indicator in a sample from the individual and predicting survival based on said knowledge. As described herein, a high intratumor heterogeneity indicator may correspond with a lower predicted survival and a low intratumor heterogeneity indicator may correspond to a higher predicted survival. In some embodiments, the individual is being treated with an anti-cancer therapy such as an endocrine therapy or a CDK4/6 inhibitor. In some embodiments, the methods comprise acquiring knowledge of aneuploidy based intratumor based heterogeneity or an intratumor heterogeneity indicator in a sample from the individual. In some instances, survival may be overall survival or progression free survival.

### Reporting

In some embodiments, the methods provided herein comprise generating a report, and/or providing a report to party.

In some embodiments, a report according to the present disclosure comprises information about one or more of aneuploidy based intratumor based heterogeneity or an intratumor heterogeneity indicator for a sample from an individual having a cancer. In some instances. the cancer is breast cancer, ovarian cancer or NSCLC. In some embodiments, the report comprises an identifier for the individual from which the sample was obtained.

In some embodiments, the report includes information on the role of aneuploidy based intratumor based heterogeneity in cancer. Such information can include one or more of: information on prognosis of a cancer, such as a cancer provided herein, information on resistance of a cancer, such as a cancer provided herein, to one or more treatments; information on potential or suggested therapeutic options (e.g., endocrine therapy and/or CDK4/6 inhibitor); or information on therapeutic options that should be avoided. In some embodiments, the report includes information on the likely effectiveness, acceptability, and/or advisability of applying a therapeutic option (e.g., endocrine therapy and/or CDK4/6 inhibitor) to an individual having a cancer characterized in the report. In some embodiments, the report includes information or a recommendation on the administration of a treatment (e.g., endocrine therapy and/or CDK4/6 inhibitor). In some embodiments, the information or recommendation includes the dosage of the treatment and/or a treatment regimen (e.g., endocrine therapy and/or CDK4/6 inhibitor). In some embodiments, the report comprises information or a recommendation for at least one, at least two, at least three, at least four, at least five, at least six, at least seven, at least eight, at least nine, at least ten, or more treatments.

Also provided herein are methods of generating a report according to the present disclosure. In some embodiments, a report according to the present disclosure is generated by a method comprising one or more of the following steps: obtaining a sample, such as a sample described herein, from an individual, e.g., an individual having a cancer, such as breast cancer, ovarian cancer or NSCLC; characterizing aneuploidy based intratumor heterogeneity in the tumor of the sample, or acquiring knowledge of the aneuploidy based intratumor heterogeneity and generating a report. In some aspects, the methods comprise generating an intratumor heterogeneity indicator and generating a report. In some embodiments, the report generated is a personalized cancer report.

A report according to the present disclosure may be in an electronic, web-based, or paper form. The report may be provided to an individual or a patient (e.g., an individual or a patient with a cancer, or to an individual or entity other than the individual or patient (e.g., other than the individual or patient with the cancer), such as one or more of a caregiver, a physician, an oncologist, a hospital, a clinic, a third party payor, an insurance company, or a government entity. In some embodiments, the report is provided or delivered to the individual or entity within any of about 1 day or more, about 7 days or more, about 14 days or more, about 21 days or more, about 30 days or more, about 45 days or more, or about 60 days or more from obtaining a sample from an individual (e.g., an individual having a cancer). In some embodiments, the report is provided or delivered to an individual or entity within any of about 1 day or more, about 7 days or more, about 14 days or more, about 21 days or more, about 30 days or more, about 45 days or more, or about 60 days or more from characterizing the aneuploidy based intratumor heterogeneity of the disclosure in a sample obtained from an individual (e.g., an individual having a cancer). In some embodiments, the report is provided or delivered to an individual or entity within any of about 1 day or more, about 7 days or more, about 14 days or more, about 21 days or more, about 30 days or more, about 45 days or more, or about 60 days or more from acquiring knowledge of the aneuploidy based intratumor heterogeneity in a sample obtained from an individual (e.g., an individual having a cancer).

The method steps of the methods described herein are intended to include any suitable method of causing one or more other parties or entities to perform the steps, unless a different meaning is expressly provided or otherwise clear from the context. Such parties or entities need not be under the direction or control of any other party or entity, and need not be located within a particular jurisdiction. Thus, for example, a description or recitation of “adding a first number to a second number” includes causing one or more parties or entities to add the two numbers together. For example, if person X engages in an arm's length transaction with person Y to add the two numbers, and person Y indeed adds the two numbers, then both persons X and Y perform the step as recited: person Y by virtue of the fact that he actually added the numbers, and person X by virtue of the fact that he caused person Y to add the numbers. Furthermore, if person X is located within the United States and person Y is located outside the United States, then the method is performed in the United States by virtue of person X's participation in causing the step to be performed.

### Subjects and Reference Subjects

In some instances, the sample is obtained (e.g., collected) from a subject or reference subject with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g., a leukemia or lymphoma. In some instances, the cancer is a solid tumor, e.g. NSCLC, ovarian cancer, or breast cancer.

In some instances, the subject or reference subject has a cancer. In some instances, the subject or reference subject is in need of being monitored for cancer progression or regression, e.g., after being treated with an anti-cancer therapy (or anti-cancer treatment). In some instances, the subject or reference subject is in need of being monitored for relapse of cancer. In some instances, the subject or reference subject is in need of being monitored for minimum residual disease (MRD). In some instances, the subject or reference subject has been, or is being treated, for cancer. In some instances, the subject or reference subject has not been treated with an anti-cancer therapy (or anti-cancer treatment).

In some instances, the subject or reference subject is being treated, or has been previously treated, with one or more targeted therapies. In some instances, the subject or reference subject who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the subject or reference subject has not been previously treated with a targeted therapy. In some instances, e.g., for a subject or reference subject who has not been previously treated with a targeted therapy, the sample comprises a resection, e.g., an original resection, or a resection following recurrence (e.g., following a disease recurrence post-therapy).

### Cancers

In some instances, the sample is acquired from a subject or reference subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), a gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMOL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Samples

The disclosed methods and systems may be used with any of a variety of samples (also referred to herein as specimens) comprising nucleic acids (e.g., DNA or RNA) that are collected from a subject or a reference subject. Examples of a sample include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavage or bronchoalveolar lavage), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) tail of mature mRNAs by priming with oligo (dT)-containing oligonucleotides. Methods for depletion, poly(A) enrichment, and cDNA synthesis are well known to those of skill in the art.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing” (or “MPS”), and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, RNA sequencing (RNAseq), low pass sequencing, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts. In some instances, sequencing may comprise RNA sequencing (RNA seq).

The disclosed methods and systems may be implemented using sequencing platforms such as the Roche/454 Genome Sequencer (GS) FLX System, Illumina/Solexa Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio® RS platform. In some instances, sequencing may comprise Illumina MiSeqÔ sequencing. In some instances, sequencing may comprise Illumina HiSeq® sequencing. In some instances, sequencing may comprise Illumina NovaSeq® sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

In some instances, identification of copy number alterations may be achieved through comparative genomic hybridization techniques (CGH) or other array based methods rather than sequencing techniques.

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing (MPS) of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147 (1): 195-197), the Striped Smith-Waterman algorithm (see, e.g., Farrar (2007), “Striped Smith-Waterman Speeds Database Searches Six Times Over Other SIMD Implementations”, Bioinformatics 23 (2): 156-161), the Needleman-Wunsch algorithm (Needleman, et al. (1970) “A General Method Applicable to the Search for Similarities in the Amino Acid Sequence of Two Proteins”, J. Molecular Biology 48 (3): 443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized.

In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. CàT in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Aneuploidy Calling

In some instances, the methods described herein may comprise the use of an aneuploidy calling method, e.g., to call aneuploidy events based on the sequence reads and fragments (complementary pairs of forward and reverse sequence reads) derived DNA. In some instances, the aneuploidy calling method may be used to determine one or more aneuploidy annotations for DNA isolated from a tumor sample. In some instances, the aneuploidy annotations may comprise gains and losses of chromosome arms. In some instances, an aneuploidy annotation is characterized as a variation in chromosome number from a base ploidy of a tumor sample. In some instances, aneuploidy annotations may be determined based on an analysis of genomic data using a method such as that described by Spurr, et al. (2021), “Quantification of Aneuploidy in Targeted Sequencing Data Using ASCETS”, Bioinformatic 2021:1-3. In some instances, the aneuploidy annotations (e.g. chromosome arm losses) may be determined based on analysis of genomic method using a method such as that described by Green, et al. (2010), “A New Method to Detect Loss of Heterozygosity Using Cohort Heterozygosity Comparisons”, BMC Cancer 10L195-203.

In some instances, the methods disclose herein may comprise detecting copy number alteration using the methods described in more detail previously, e.g., International Patent Application Publication No. WO2023/60250, the entire content of which is incorporated herein by reference. In some instances, the methods disclosed herein may comprise automated calling of copy number alterations using the methods described in more detail previously, e.g., International Patent Application Publication No WO2023/196390.

In some instances, the methods described herein may comprise the use of an aneuploidy calling method, e.g., to call aneuploidy events based on the sequence reads and fragments (complementary pairs of forward and reverse sequence reads) derived RNA. In some instances, the aneuploidy calling method may be used to determine one or more aneuploidy annotations for RNA isolated from a tumor sample. In some instances, an aneuploidy annotation is characterized as a variation in gene expression compared to gene expression from a base ploidy of a tumor sample. In some instances, gain or loss of chromosomal portions may be determined by detecting increases or decreases in expression for genes in the chromosomal portion of interest.

In some instances, any aneuploidy calling method may be used to determine one or more aneuploidy annotations in DNA isolated from a sample. In some instances, an aneuploidy annotation may be characterized as an arm level chromosomal gain and/or arm level chromosomal loss. In some instances, the chromosome arm may be 1p, 1q, 2p, 2q, 3p, 3q, 4p, 4q, 5p, 5q, 6p, 6q, 7p, 7q, 8p, 7q, 8p, 8q, 9p, 9q, 10p, 10q, 11p, 11q, 12p, 12q, 13p, 13q, 14p, 14q, 15p, 15q, 16p, 16q, 17p, 17q, 18p, 18q, 19p, 19q, 20p, 20q, 21p, 21q, 22p, 22q, Xp, Xq, Yp, or Yq. In some instances, an aneuploidy annotation be characterized as a cytoband gain or cytoband loss. In some instances, a cytoband may be named according the International System for Human Cytogenomic Nomenclature.

### Systems

Also disclosed herein are systems designed to implement any of the disclosed methods which can be used for characterizing aneuploidy based intratumor heterogeneity in a sample from a subject. The systems may comprise, e.g., one or more processors, and a memory unit communicatively coupled to the one or more processors and configured to store instructions that, when executed by the one or more processors, cause the system to: take in sample aneuploidy data for the tumor; call subclonal aneuploidy events in the sample aneuploidy data; wherein a subject aneuploidy event is characterized as subclonal based on a comparison of the subject aneuploidy event to a corresponding reference aneuploidy event for the tumor type; wherein the reference aneuploidy event had been characterized by: obtaining reference aneuploidy data for a plurality of reference tumor samples of the tumor type; wherein the plurality of reference tumor samples comprises at least two tumor samples obtained at different time points from each reference subject in a plurality of reference subjects; characterizing, for each aneuploidy event in a plurality of aneuploidy events, as unique or shared among the at least two tumor samples from each reference subject in the plurality of reference subjects; determining, for each aneuploidy event in the plurality of aneuploidy events; whether uniqueness of the aneuploidy event within the plurality of aneuploid events is enriched compared to uniqueness of all aneuploidy events within the plurality of aneuploidy events, and characterizing the aneuploidy event as significantly subclonal or significantly clonal based on enrichment of the uniqueness of the aneuploidy event; and generate an intratumor heterogeneity score for the tumor sample based on a number of called significantly subclonal events in the sample aneuploidy data for the tumor sample.

In some instances, the disclosed systems may further comprise a sequencer, e.g., a next generation sequencer (also referred to as a massively parallel sequencer). Examples of next generation (or massively parallel) sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLID) system, Polonator's G.007 system, Helicos BioSciences' HeliScope Gene Sequencing system, ThermoFisher Scientific's Ion Torrent Genexus system, or Pacific Biosciences' PacBio® RS system.

In some instances, the disclosed systems may be used for characterizing aneuploidy based intratumor heterogeneity for a tumor of a tumor type from a subject in any of a variety of samples as described herein (e.g., a tissue sample, biopsy sample, hematological sample, or liquid biopsy sample derived from the subject).

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the determination of an aneuploidy based intratumor heterogeneity score for a tumor is used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Machine Learning

Any of a variety of machine learning approaches & algorithms (where a machine learning model, as referred to herein, comprises a trained machine learning algorithm) may be used in implementing the disclosed methods. For example, the machine learning model may comprise a supervised learning model (i.e., a model trained using labeled sets of training data), an unsupervised learning model (i.e., a model trained using unlabeled sets of training data), a semi-supervised learning model (i.e., a model trained using a combination of labeled and unlabeled training data), a self-supervised learning model, or any combination thereof. In some examples, the machine learning model can comprise a deep learning model (i.e., a model comprising many layers of coupled “nodes” that may be trained in a supervised, unsupervised, or semi-supervised manner).

In some instances, one or more machine learning models (e.g., 1, 2, 3, 4, 5, 6, 7, 8, 9, 10, or more than 10 machine learning models), or a combination thereof, may be utilized to implement the disclosed methods.

In some instances, the one or more machine learning models may comprise statistical methods for analyzing data. The machine learning models may be used for classification and/or regression of data. The machine learning models can include, for example, neural networks, support vector machines, decision trees, ensemble learning (e.g., bagging-based learning, such as random forest, and/or boosting-based learning), k-nearest neighbors algorithms, linear regression-based models, and/or logistic regression-based models. The machine learning models can comprise regularization, such as L1 regularization and/or L2 regularization. The machine learning models can include the use of dimensionality reduction techniques (e.g., principal component analysis, matrix factorization techniques, and/or autoencoders) and/or clustering techniques (e.g., hierarchical clustering, k-means clustering, distribution-based clustering, such as Gaussian mixture models, or density-based clustering, such as DBSCAN or OPTICS). The one or more machine learning models can comprise solving, e.g., optimizing, an objective function over multiple iterations based on a training data set. The iterative solving approach can be used even when the machine learning model comprises a model for which there exists a closed-form solution (e.g., linear regression).

In some instances, the machine learning models can comprise artificial neural networks (ANNs), e.g., deep learning models. For example, the one or more machine learning models/algorithms used for implementing the disclosed methods may include an ANN which can comprise any of a variety of computational motifs/architectures known to those of skill in the art, including, but not limited to, feedforward connections (e.g., skip connections), recurrent connections, fully connected layers, convolutional layers, and/or pooling functions (e.g., attention, including self-attention). The artificial neural networks can comprise differentiable non-linear functions trained by backpropagation.

Artificial neural networks, e.g., deep learning models, generally comprise an interconnected group of nodes organized into multiple layers of nodes. For example, the ANN architecture may comprise at least an input layer, one or more hidden layers (i.e., intermediate layers), and an output layer. The ANN or deep learning model may comprise any total number of layers (e.g., 3, 4, 5, 6, 7, 8, 9, 10, 15, 20, or more than 20 layers in total), and any number of hidden layers (e.g., 1, 2, 3, 4, 5, 6, 7, 8, 9, 10, 15, 20, or more than 20 hidden layers), where the hidden layers function as trainable feature extractors that allow mapping of a set of input data to a preferred output value or set of output values. Each layer of the neural network comprises a plurality of nodes (e.g., at least 10, 25, 50, 75 100, 200, 300, 400, 500, 600, 700, 800, 900, 1000, 2000, 3000, 4000, 5000, 6000, 7000, 8000, 9000, 10,000, or more than 10,000 nodes). A node receives input data (e.g., genomic feature data (such as variant sequence data, methylation status data, etc.), non-genomic feature data (e.g., digital pathology image feature data), or other types of input data (e.g., patient-specific clinical data)) that comes either directly from one or more input data nodes or from the output of one or more nodes in previous layers, and performs a specific operation, e.g., a summation operation. In some cases, a connection from an input to a node is associated with a weight (or weighting factor). In some cases, the node may, for example, sum up the products of all pairs of inputs, Xᵢ, and their associated weights, Wᵢ. In some cases, the weighted sum is offset with a bias, b. In some cases, the output of a node may be gated using a threshold or activation function, ƒ, where ƒ may be a linear or non-linear function. The activation function may be, for example, a rectified linear unit (ReLU) activation function or other function such as a saturating hyperbolic tangent, identity, binary step, logistic, arcTan, softsign, parameteric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network (or other machine learning architecture), can be “taught” or “learned” in a training phase using one or more sets of training data (e.g., 1, 2, 3, 4, 5, or more than 5 sets of training data) and a specified training approach configured to solve, e.g., minimize, a loss function. For example, the adjustable parameters for an ANN (e.g., deep learning model) may be determined based on input data from a training data set using an iterative solver (such as a gradient-based method, e.g., backpropagation), so that the output value(s) that the ANN computes (e.g., a classification of a sample or a prediction of a disease outcome) are consistent with the examples included in the training data set. The training of the model (i.e., determination of the adjustable parameters of the model using an iterative solver) may or may not be performed using the same hardware as that used for deployment of the trained model.

In some instances, the disclosed methods may comprise retraining any of the machine learning models (e.g., iteratively retraining a previously trained model using one or more training data sets that differ from those used to train the model initially). In some instances, retraining the machine learning model may comprise using a continuous, e.g., online, machine learning model, i.e., where the model is periodically or continuously updated or retrained based on new training data. The new training data may be provided by, e.g., a single deployed local operational system, a plurality of deployed local operational systems, or a plurality of deployed, geographically-distributed operational systems. In some instances, the disclosed methods may employ, for example, pre-trained ANNs, and the pre-trained ANNs can be fine-tuned according to an additional dataset that is inputted into the pre-trained ANN.

### Computer Systems and Networks

The processes shown in FIG. 2 can be performed, for example, using one or more electronic devices implementing a software platform. In some examples, processes 202-206 are performed using a client-server system, and the blocks of processes 202-206 are divided up in any manner between the server and a client device. In other examples, the blocks of processes 202-206 are divided up between the server and multiple client devices. Thus, while portions of processes 202-206 are described herein as being performed by particular devices of a client-server system, it will be appreciated that processes 202-206 are not so limited. In other examples, processes 202-206 are performed using only a client device or only multiple client devices. In processes 202-206 some blocks are, optionally, combined, the order of some blocks is, optionally, changed, and some blocks are, optionally, omitted. In some examples, additional steps may be performed in combination with the processes 202-206. Accordingly, the operations as illustrated (and described in greater detail below) are exemplary by nature and, as such, should not be viewed as limiting.

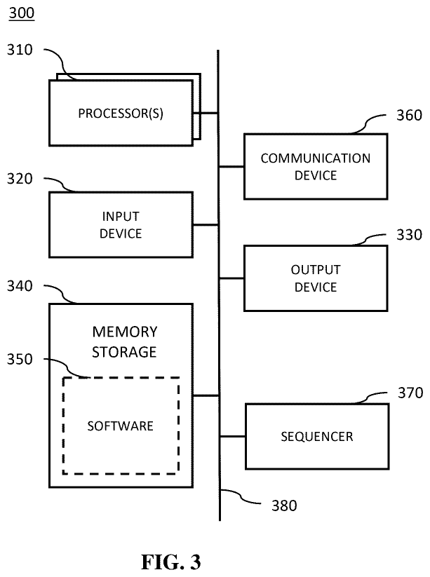

FIG. 3 illustrates an example of a computing device or system in accordance with one embodiment. Device 300 can be a host computer connected to a network. Device 600 can be a client computer or a server. As shown in FIG. 3, device 300 can be any suitable type of microprocessor-based device, such as a personal computer, workstation, server or handheld computing device (portable electronic device) such as a phone or tablet. The device can include, for example, one or more processor(s) 310, input devices 320, output devices 330, memory or storage devices 340, communication devices 360, and nucleic acid sequencers 370. Software 350 residing in memory or storage device 340 may comprise, e.g., an operating system as well as software for executing the methods described herein. Input device 320 and output device 330 can generally correspond to those described herein, and can either be connectable or integrated with the computer.

Input device 320 can be any suitable device that provides input, such as a touch screen, keyboard or keypad, mouse, or voice-recognition device. Output device 330 can be any suitable device that provides output, such as a touch screen, haptics device, or speaker.

Storage 340 can be any suitable device that provides storage (e.g., an electrical, magnetic or optical memory including a RAM (volatile and non-volatile), cache, hard drive, or removable storage disk). Communication device 360 can include any suitable device capable of transmitting and receiving signals over a network, such as a network interface chip or device. The components of the computer can be connected in any suitable manner, such as via a wired media (e.g., a physical system bus 380, Ethernet connection, or any other wire transfer technology) or wirelessly (e.g., Bluetooth®, Wi-Fi®, or any other wireless technology).

Software module 350, which can be stored as executable instructions in storage 340 and executed by processor(s) 310, can include, for example, an operating system and/or the processes that embody the functionality of the methods of the present disclosure (e.g., as embodied in the devices as described herein).

Software module 350 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 640, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 350 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

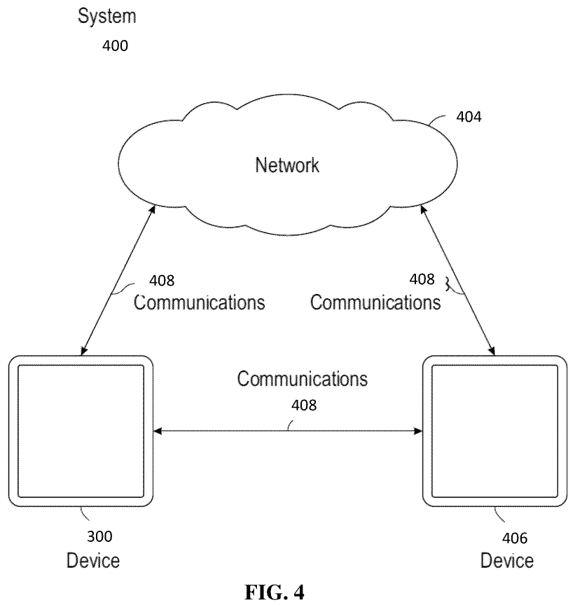

Device 300 may be connected to a network (e.g., network 404, as shown in FIG. 4 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 300 can be implemented using any operating system, e.g., an operating system suitable for operating on the network. Software module 350 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 310.

Device 300 can further include a sequencer 370, which can be any suitable nucleic acid sequencing instrument.

FIG. 4 illustrates an example of a computing system in accordance with one embodiment. In system 400, device 300 (e.g., as described above and illustrated in FIG. 3) is connected to network 404, which is also connected to device 406. In some embodiments, device 406 is a sequencer. Exemplary sequencers can include, without limitation, Roche/454's Genome Sequencer (GS) FLX System, Illumina/Solexa's Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLID) system, Polonator's G.007 system, Helicos BioSciences' HeliScope Gene Sequencing system, or Pacific Biosciences' PacBio® RS system.

Devices 300 and 406 may communicate, e.g., using suitable communication interfaces via network 404, such as a Local Area Network (LAN), Virtual Private Network (VPN), or the Internet. In some embodiments, network 1004 can be, for example, the Internet, an intranet, a virtual private network, a cloud network, a wired network, or a wireless network. Devices 300 and 406 may communicate, in part or in whole, via wireless or hardwired communications, such as Ethernet, IEEE 802.11b wireless, or the like. Additionally, devices 300 and 406 may communicate, e.g., using suitable communication interfaces, via a second network, such as a mobile/cellular network. Communication between devices 300 and 406 may further include or communicate with various servers such as a mail server, mobile server, media server, telephone server, and the like. In some embodiments, Devices 300 and 406 can communicate directly (instead of, or in addition to, communicating via network 404), e.g., via wireless or hardwired communications, such as Ethernet, IEEE 802.11b wireless, or the like. In some embodiments, devices 300 and 406 communicate via communications 408, which can be a direct connection or can occur via a network (e.g., network 404).

One or all of devices 300 and 406 generally include logic (e.g., http web server logic) or are programmed to format data, accessed from local or remote databases or other sources of data and content, for providing and/or receiving information via network 404 according to various examples described herein.

### Exemplary Embodiments

The following embodiments are exemplary and are not intended to limit the scope of the invention described herein and as recited in the claims. Exemplary embodiments of the methods and systems described herein include:

Embodiment 1. A method of characterizing aneuploidy based intratumor heterogeneity for a tumor of a tumor type from a subject, comprising: obtaining sample aneuploidy data for the tumor; calling subclonal aneuploidy events in the sample aneuploidy data, wherein a subject aneuploidy event is characterized as subclonal based on a comparison of the subject aneuploidy event to a corresponding reference aneuploidy event for the tumor type, wherein the reference aneuploidy event had been characterized by: obtaining reference aneuploidy data for a plurality of reference tumor samples of the tumor type, wherein the plurality of reference tumor samples comprises at least two tumor samples obtained at different time points from each reference subject in a plurality of reference subjects, characterizing, for each aneuploidy event in a plurality of aneuploidy events, as unique or shared among the at least two tumor samples from each reference subject in the plurality of reference subjects, determining, for each aneuploidy event in the plurality of aneuploidy events, whether uniqueness of the aneuploidy event within the plurality of aneuploid events is enriched compared to uniqueness of all aneuploidy events within the plurality of aneuploidy events, and characterizing the aneuploidy event as significantly subclonal or significantly clonal based on enrichment of the uniqueness of the aneuploidy event; and generating an intratumor heterogeneity score for the tumor sample based on a number of called significantly subclonal events in the sample aneuploidy data for the tumor sample.

Embodiment 2. A method of characterizing significantly subclonal events or significantly clonal events of a tumor type, comprising: obtaining reference aneuploidy data for a plurality of reference tumor samples of the tumor type, wherein the plurality of reference tumor samples comprises at least two tumor samples obtained at different time points from each reference subject in a plurality of reference subjects, characterizing, for each aneuploidy event in a plurality of aneuploidy events, as unique or shared among the at least two tumor samples from each reference subject in the plurality of reference subjects, determining, for each aneuploidy event in the plurality of aneuploidy events, whether uniqueness of the aneuploidy event within the plurality of aneuploid events is enriched compared to uniqueness of all aneuploidy events within the plurality of aneuploidy events, and characterizing the aneuploidy event as significantly subclonal or significantly clonal based on enrichment of the uniqueness of the aneuploidy event.

Embodiment 3. The method of embodiment 1, wherein the sample aneuploidy data comprises one or more aneuploidy event annotations detected in a single sample collected from the tumor.

Embodiment 4. The method of any of embodiments 1-3, wherein the reference aneuploidy data comprises one or more aneuploidy event annotations for the plurality of reference tumor samples of the tumor type.

Embodiment 5. The method of any of embodiments 1-4, wherein the one or more aneuploidy event annotations are characterized as a variation in chromosome number from a base ploidy of the sample.

Embodiment 6. The method of embodiment 4 or embodiment 5, wherein the one or more aneuploidy event annotations comprise a plurality of aneuploidy events.

Embodiment 7. The method of embodiment 6, wherein an aneuploidy event in the plurality of aneuploidy events is a gain of a chromosomal portion or a loss of a chromosomal portion.

Embodiment 8. The method of embodiment 7, wherein a chromosomal portion is a chromosomal arm.

Embodiment 9. The method of embodiment 7, wherein a chromosomal portion is a cytoband.

Embodiment 10. The method of any of embodiments 1-9, wherein the tumor type is non-small cell lung cancer (NSCLC), or ovarian cancer.

Embodiment 11. The method of any of embodiments 1-9, wherein the tumor type is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

Embodiment 12. The method of any of embodiments 1-9, wherein the tumor type comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

Embodiment 13. The method of any of embodiments 1-12, wherein determining whether uniqueness of an aneuploidy event within the plurality of aneuploid events is enriched compared to uniqueness of all aneuploidy events within the plurality of aneuploidy events comprises comparing how often the aneuploidy event is subclonal, performing a Fisher's exact test, or performing a chi-squared test.

Embodiment 14. The method of embodiment 13, wherein performing the Fisher's exact test comprises generating an odds ratio.

Embodiment 15. The method of embodiment 14, wherein an aneuploidy event is significantly subclonal if the fold change in odds ratio is beyond a cutoff in the negative direction.

Embodiment 16. The method of embodiment 15, wherein the cutoff is more negative than about −1.5.

Embodiment 17. The method of embodiment 15, wherein an aneuploidy event is significantly clonal if the fold change in odds ratio beyond a cutoff in the positive direction.

Embodiment 18. The method of embodiment 17, wherein the cutoff is greater than about 1.5.

Embodiment 19. The method of any of embodiment 1 and embodiments 3-18, wherein obtaining sample aneuploidy data comprises; performing a tumor biopsy; extracting tumor nucleic acids; sequencing, by a sequencer, the extracted tumor nucleic acids; receiving a tumor sequence data from the sequencers; and providing the tumor sequence data to a program configured to receive tumor sequence data and identify a plurality of aneuploidy annotations.

Embodiment 20. The method of embodiment 19, wherein the tumor biopsy is a liquid biopsy and comprises cell-free DNA (cfDNA), circulating tumor DNA (ctDNA), RNA or any combination thereof.

Embodiment 21. The method of embodiment 20, wherein the liquid biopsy comprises blood, plasma, cerebrospinal fluid, sputum, stool, urine, or saliva.

Embodiment 22. The method of any of embodiments 19-21, wherein extracting tumor nucleic acids comprises extracting ctDNA.

Embodiment 23. The method of any of embodiments 19-22, wherein the sequencing comprises use of a massively parallel sequencing (MPS) technique, whole genome sequencing (WGS), RNA sequencing (RNAseq), low pass sequencing, whole exome sequencing, targeted sequencing, direct sequencing, or Sanger sequencing technique.

Embodiment 24. The method of embodiment 23, wherein the sequencing comprises massively parallel sequencing, and the massively parallel sequencing technique comprises next generation sequencing (NGS).

Embodiment 25. The method of embodiment 24, wherein the sequencer comprises a next generation sequencer.

Embodiment 26. The method of any of embodiment 1, or embodiments 3-25, wherein generating the intratumor heterogeneity score comprises summing the number of called significantly subclonal events in the sample aneuploidy event data for the tumor sample.

Embodiment 27. The method of any of embodiment 1, or embodiments 3-26, further comprising generating an intratumor heterogeneity indicator, wherein the intratumor heterogeneity indicator relates to the relationship between the intratumor heterogeneity score and a determined threshold.

Embodiment 28. The method of embodiment 27, wherein the determined threshold comprises an upper threshold and a lower threshold.

Embodiment 29. The method of embodiment 28, wherein the intratumor heterogeneity indicator is high if the intratumor heterogeneity metric is greater than or equal to the upper threshold, the intratumor heterogeneity indicator is intermediate if the intratumor heterogeneity metric is greater than the lower threshold and less than the lower threshold, and the intratumor heterogeneity indicator is low if intratumor heterogeneity metric is less than or equal to the lower threshold.

Embodiment 30. The method of embodiment 29, wherein the high intratumor heterogeneity score relates to poor prognosis, quick resistance to cancer therapies, or poor outcomes.

Embodiment 31. The method of any of embodiment 1 and embodiments 3-30, further comprising generating an aneuploidy burden score by integrating the intratumor heterogeneity score with digital pathology-based heterogeneity, single cell heterogeneity scores, radiological heterogeneity scores, aneuploidy burden, cytoband features, CN segment features for the tumor.

Embodiment 32. A method of selecting a treatment for an individual with cancer, comprising: (a) characterizing aneuploidy based intratumor heterogeneity in a sample from the individual according to the methods of any of embodiments 1 and 3-31; and (b) selecting a treatment based on the aneuploidy based intratumor heterogeneity.

Embodiment 33. A method of treating or delaying progression of cancer in an individual, comprising: (a) characterizing aneuploidy based intratumor heterogeneity in a sample from the individual according to the methods of any of embodiments 1 and 3-31; and (b) administering to the individual an effective amount of a therapy based on the intratumor heterogeneity.

Embodiment 34. A method of predicting survival of an individual having cancer, comprising acquiring knowledge of an intratumor heterogeneity indicator in a sample from the individual, wherein responsive to the acquisition of said knowledge, the individual is predicted to have longer survival when the intratumor heterogeneity indicator is low than if the intratumor heterogeneity indicator is high.

Embodiment 35. The method of embodiment 34, wherein acquiring knowledge of an intratumor heterogeneity indicators comprises generating an intratumor heterogeneity indicator according to the method of any one of embodiments 27-31.

Embodiment 36. A method, comprising: obtaining sample aneuploidy data for a non-small cell lung cancer (NSCLC) tumor of a subject; calling subclonal aneuploidy events in the sample aneuploidy data, wherein a subject aneuploidy event is characterized as subclonal based on a comparison of the subject aneuploidy event to a corresponding NSCLC reference aneuploidy event on a list of NSCLC significantly subclonal events; and generating an intratumor heterogeneity score based on a number of aneuploidy events in the aneuploidy event data on the list of NSCLC significantly subclonal events.

Embodiment 37. The method of embodiment 36, wherein the list of NSCLC significantly subclonal events comprise a plurality of NSCLC reference aneuploidy events.

Embodiment 38. The method of embodiment 37 wherein the plurality of NSCLC reference aneuploidy events comprise arm level chromosome gains of 2p, 2q, 3p, 4q, 6q, 10q, 12q, 13q, 15q, 16q, 17p, 18q, 19p, 21q, and 22q, and arm level chromosomal losses of 1q, 2p, 2q, 3q, 5p, 6p, 7p, 7q, 11p, 11q, 12q, 16p, 17q, and 20q.

Embodiment 39. The method of any of embodiments 36-38, wherein the sample aneuploidy data comprises one or more aneuploidy event annotations.

Embodiment 40. The method of any of embodiments 36-39, wherein the one or more aneuploidy event annotations are characterized as a variation in chromosome number from a base ploidy of the sample.

Embodiment 41. The method of embodiment 39 or embodiment 40, wherein the one or more aneuploidy event annotations comprise a plurality of aneuploidy events.

Embodiment 42. The method of embodiment 41, wherein an aneuploidy event in the plurality of aneuploidy events is an arm level chromosome gain or an arm level chromosomal loss.

Embodiment 43. The method of any of embodiments 36-42, wherein generating the intratumor heterogeneity score comprises summing the number of called significantly subclonal events in the sample aneuploidy event data for the tumor sample.

Embodiment 44. The method of any of embodiments 36-43, further comprising generating an intratumor heterogeneity indicator, wherein the intratumor heterogeneity indicator relates to the relationship between the intratumor heterogeneity score and a determined threshold.

Embodiment 45. The method of embodiment 44, wherein a determined threshold comprises an upper threshold and a lower threshold.

Embodiment 46. The method of embodiment 45, wherein the intratumor heterogeneity indicator is high if the intratumor heterogeneity metric is greater or equal to the upper threshold, the intratumor heterogeneity indicator is intermediate if the intratumor heterogeneity metric is greater than the lower threshold and less than the lower threshold, and the intratumor heterogeneity indicator is low if intratumor heterogeneity metric is less than or equal to the lower threshold.

Embodiment 47. The method of embodiment 46, wherein the upper threshold is between 2 and 6.

Embodiment 48. The method of embodiment 46 or embodiment 47, wherein the lower threshold is between 0 and 2,

Embodiment 49. The method embodiment 46, wherein the upper threshold is 4 and the lower threshold is 1.

Embodiment 50. The method of any of embodiments 46-49, wherein a high intratumor heterogeneity score relates to poor prognosis, quick resistance to cancer therapies, or poor outcomes.

Embodiment 51. The method of any of embodiments 36-50, wherein obtaining sample aneuploidy data comprises; performing a tumor biopsy; extracting tumor nucleic acids; sequencing, by a sequencer, the extracted tumor nucleic acids; receiving a tumor sequence data from the sequencers; and providing the tumor sequence data to a program configured to receive tumor sequence data and identify a plurality of aneuploidy annotations.

Embodiment 52. The method of embodiment 51, wherein the tumor biopsy is a liquid biopsy and comprises cell-free DNA (cfDNA), circulating tumor DNA (ctDNA), RNA or any combination thereof.

Embodiment 53. The method of embodiment 52, wherein the sample is a liquid biopsy sample and comprises blood, plasma, cerebrospinal fluid, sputum, stool, urine, or saliva.

Embodiment 54. The method of any of embodiment 52 or embodiment 53, wherein extracting tumor nucleic acids comprises extracting ctDNA.

Embodiment 55. The method of any of embodiments 41-54, wherein the sequencing comprises use of a massively parallel sequencing (MPS) technique, RNA sequencing (RNAseq), low pass sequencing, whole genome sequencing (WGS), whole exome sequencing, targeted sequencing, direct sequencing, or Sanger sequencing technique.

Embodiment 56. The method of embodiment 55, wherein the sequencing comprises massively parallel sequencing, and the massively parallel sequencing technique comprises next generation sequencing (NGS).

Embodiment 57. The method of embodiment 56, wherein the sequencer comprises a next generation sequencer.

Embodiment 58. A method, comprising: obtaining sample aneuploidy data for an ovarian tumor of a subject; calling subclonal events in the sample aneuploidy data, wherein a subject aneuploidy event is characterized as subclonal based on a comparison of the subject aneuploidy event to a corresponding ovarian reference aneuploidy event on a list of ovarian significantly subclonal events; and generating an intratumor heterogeneity score based on a number of aneuploidy events in the aneuploidy event data on a list of ovarian significantly subclonal events.

Embodiment 59. The method of embodiment 58, wherein the list of ovarian significantly subclonal events comprise a plurality of ovarian reference aneuploidy events.

Embodiment 60. The method of embodiment 59, the plurality of ovarian reference aneuploidy events comprise arm level chromosome gains of 1p, 3p, 4q, 11p, 12q, 13q, 16p, 16q, 17q, 19q, 21q and 22q, and arm level chromosomal losses of 1q, 2p, 2q, 3p, 5p, 6p, 7q, 8q, 10p, 12q, 17q, 20p, 20q, and 21q.

Embodiment 61. The method of embodiment 59 or embodiment 60, wherein the sample aneuploidy data comprises one or more aneuploidy event annotations.

Embodiment 62. The method of any of embodiments 58-61, wherein the reference aneuploidy data comprises one or more aneuploidy event annotations for the plurality of reference ovarian tumor samples.

Embodiment 63. The method of any of embodiments 58-62, wherein the one or more aneuploidy event annotations are characterized as a variation in chromosome number from a base ploidy of the sample.

Embodiment 64. The method of embodiment 62 or embodiment 63, wherein the one or more aneuploidy event annotations comprise a plurality of aneuploidy events.

Embodiment 65. The method of embodiment 64, wherein an aneuploidy event in the plurality of aneuploidy events is an arm level chromosome gain or an arm level chromosomal loss.

Embodiment 66. The method of any of embodiments 58-65, wherein generating the intratumor heterogeneity score comprises summing the number of called significantly subclonal events in the sample aneuploidy event data for the tumor sample.

Embodiment 67. The method of any of embodiments 58-66, further comprising generating an intratumor heterogeneity indicator, wherein the intratumor heterogeneity indicator relates to the relationship between the intratumor heterogeneity score and a determined threshold.

Embodiment 68. The method of embodiment 67, wherein a determined threshold comprises an upper threshold and a lower threshold.

Embodiment 69. The method of embodiment 68, wherein the intratumor heterogeneity indicator is high if the intratumor heterogeneity metric is greater than or equal to the upper threshold, the intratumor heterogeneity indicator is intermediate if the intratumor heterogeneity metric is greater than the lower threshold and less than the lower threshold, and the intratumor heterogeneity indicator is low if intratumor heterogeneity metric is less than or equal to the lower threshold.

Embodiment 70. The method of embodiment 68 or embodiment 69, wherein the upper threshold is between 2 and 6.

Embodiment 71. The method of any of embodiments 68-70, wherein the lower threshold is between 0 and 2,

Embodiment 72. The method embodiment 68 or embodiment 69, wherein the upper threshold is 4 and the lower threshold is 1.

Embodiment 73. The method of any of embodiments 69-72, wherein a high intratumor heterogeneity score relates to poor prognosis, quick resistance to cancer therapies, or poor outcomes.

Embodiment 74. The method of any of embodiments 58-73, wherein obtaining sample aneuploidy data comprises; performing a tumor biopsy; extracting tumor nucleic acids; sequencing, by a sequencer, the extracted tumor nucleic acids; receiving a tumor sequence data from the sequencers; and providing the tumor sequence data to a program configured to receive tumor sequence data and identify a plurality of aneuploidy annotations.

Embodiment 75. The method of embodiment 74, wherein the tumor biopsy is a liquid biopsy and comprises cell-free DNA (cfDNA), circulating tumor DNA (ctDNA), RNA, or any combination thereof.

Embodiment 76. The method of embodiment 75, wherein the sample is a liquid biopsy sample and comprises blood, plasma, cerebrospinal fluid, sputum, stool, urine, or saliva.

Embodiment 77. The method of any of embodiment 74 or embodiment 75, wherein extracting tumor nucleic acids comprises extracting ctDNA.

Embodiment 78. The method of any of embodiments 74-77, wherein the sequencing comprises use of a massively parallel sequencing (MPS) technique, whole genome sequencing (WGS), RNA sequencing (RNAseq), low pass sequencing, whole exome sequencing, targeted sequencing, direct sequencing, or Sanger sequencing technique.

Embodiment 79. The method of embodiment 78, wherein the sequencing comprises massively parallel sequencing, and the massively parallel sequencing technique comprises next generation sequencing (NGS).

Embodiment 80. The method of embodiment 79, wherein the sequencer comprises a next generation sequencer.

Embodiment 81. A method, comprising: obtaining sample aneuploidy data for a breast tumor of a subject; calling subclonal events in the sample aneuploidy data, wherein a subject aneuploidy event is characterized as subclonal based on a comparison of the subject aneuploidy event to a corresponding breast cancer reference aneuploidy event on a list of breast cancer significantly subclonal events; andgenerating an intratumor heterogeneity score based on a number of aneuploidy events in the aneuploidy event data on a list of breast cancer significantly subclonal events.

Embodiment 82. The method of embodiment 81, wherein the list of breast cancer significantly subclonal events comprise a plurality of breast cancer reference aneuploidy events.

Embodiment 83. The method of embodiment 82, the plurality of breast cancer reference aneuploidy events comprise arm level chromosome gains of 1p, 2p, 2q, 3p, 4p, 4q, 9q, 10q, 11p, 11q, 13q, 14q, 15q, 16q, 17p, 18p, 18q, 19p, 19q, 21q and 22q, and arm level chromosomal losses of 1p, 2p, 2q, 3q, 5p, 6p, 7p, 8q, 10p, 16p, 19p, 19q, 20p, and 20q.

Embodiment 84. The method of embodiment 82 or embodiment 83, wherein the sample aneuploidy data comprises one or more aneuploidy event annotations.

Embodiment 85. The method of any of embodiments 81-84, wherein the reference aneuploidy data comprises one or more aneuploidy event annotations for the plurality of reference breast tumor samples.

Embodiment 86. The method of any of embodiments 81-85, wherein the one or more aneuploidy event annotations are characterized as a variation in chromosome number from a base ploidy of the sample.

Embodiment 87. The method of embodiment 85 or embodiment 86, wherein the one or more aneuploidy event annotations comprise a plurality of aneuploidy events.

Embodiment 88. The method of embodiment 87, wherein an aneuploidy event in the plurality of aneuploidy events is an arm level chromosome gain or an arm level chromosomal loss.

Embodiment 89. The method of any of embodiments 81-88, wherein generating the intratumor heterogeneity score comprises summing the number of called significantly subclonal events in the sample aneuploidy event data for the breast tumor sample.

Embodiment 90. The method of any of embodiments 81-89, further comprising generating an intratumor heterogeneity indicator, wherein the intratumor heterogeneity indicator relates to the relationship between the intratumor heterogeneity score and a determined threshold.

Embodiment 91. The method of embodiment 90, wherein a determined threshold comprises an upper threshold and a lower threshold.

Embodiment 92. The method of embodiment 91, wherein the intratumor heterogeneity indicator is high if the intratumor heterogeneity metric is greater than or equal to the upper threshold, the intratumor heterogeneity indicator is intermediate if the intratumor heterogeneity metric is greater than the lower threshold and less than the lower threshold, and the intratumor heterogeneity indicator is low if intratumor heterogeneity metric is less than or equal to the lower threshold.

Embodiment 93. The method of embodiment 91 or embodiment 92, wherein the upper threshold is between 1 and 5.

Embodiment 94. The method of any of embodiments 91-93, wherein the lower threshold is between 0 and 1.

Embodiment 95. The method embodiment 91 or embodiment 92, wherein the upper threshold is 1 and the lower threshold is 5.

Embodiment 96. The method of any of embodiments 92-95, wherein a high intratumor heterogeneity score relates to poor prognosis, quick resistance to cancer therapies, or poor outcomes.

Embodiment 97. The method of embodiment 96, wherein the poor outcomes comprises a shorter survival.

Embodiment 98. The method of embodiment 97, wherein the survival is progression free survival.

Embodiment 99. The method of any of embodiments 81-98, wherein obtaining sample aneuploidy data comprises; performing a tumor biopsy; extracting tumor nucleic acids; sequencing, by a sequencer, the extracted tumor nucleic acids; receiving a tumor sequence data from the sequencers; and providing the tumor sequence data to a program configured to receive tumor sequence data and identify a plurality of aneuploidy annotations.

Embodiment 100. The method of embodiment 99, wherein the tumor biopsy is a liquid biopsy and comprises cell-free DNA (cfDNA), circulating tumor DNA (ctDNA), RNA, or any combination thereof.

Embodiment 101. The method of embodiment 100, wherein the sample is a liquid biopsy sample and comprises blood, plasma, cerebrospinal fluid, sputum, stool, urine, or saliva.

Embodiment 102. The method of embodiment 100 or embodiment 101, wherein extracting tumor nucleic acids comprises extracting ctDNA.

Embodiment 103. The method of any of embodiments 99-102, wherein the sequencing comprises use of a massively parallel sequencing (MPS) technique, whole genome sequencing (WGS), RNA sequencing (RNAseq), low pass sequencing, whole exome sequencing, targeted sequencing, direct sequencing, or Sanger sequencing technique.

Embodiment 104. The method of embodiment 103, wherein the sequencing comprises massively parallel sequencing, and the massively parallel sequencing technique comprises next generation sequencing (NGS).

Embodiment 105. The method of embodiment 104, wherein the sequencer comprises a next generation sequencer.

Embodiment 106. The method of any of embodiments 81-105, wherein the subject is diagnosed with stage 1 breast cancer.

Embodiment 107. The method of any of embodiments 81-106, wherein the subject received a breast cancer therapy.

Embodiment 108. The method of embodiment 107, wherein the breast cancer therapy comprises a CDK4/6 inhibitor and an endocrine therapy in a first-line metastatic setting.

Embodiment 109. A method of predicting survival of an individual having breast cancer, comprising acquiring knowledge of an intratumor heterogeneity indicator in a sample from the individual, wherein responsive to the acquisition of said knowledge, the individual is predicted to have longer survival when the intratumor heterogeneity indicator is low than if the intratumor heterogeneity indicator is high.

Embodiment 110. The method of claim 109, wherein acquiring knowledge of an intratumor heterogeneity indicators comprises generating an intratumor heterogeneity indicator according to the method of any one of claims 90-108.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

### EXAMPLES

The following examples are included for illustrative purposes only and are not intended to limit the scope of the present disclosure.

### Example 1—Longitudinal Biopsies to Determine High-Confidence Clonal Vs Subclonal Chromosome Arm Level Events in Non-Small Cell Lung Cancer (NSCLC)

Longitudinal tumor biopsies from NSCLC patients were examined to identify significantly clonal and subclonal chromosome arm level events. The events were used to test for intratumor heterogeneity (ITH) in NSCLC tumor biopsies.

Comprehensive genomic profiling of DNA extracted from NSCLC patient samples were performed during routine clinical care (N=106,149). The comprehensive genomic profile included chromosomal arm-level annotations. Copy number modeling was used to calculate tumor purity and samples with less than 30% purity were removed from the dataset. The remaining samples (N=51,911) were filtered to data originating from patients with genomic profiling data from 2 or more biopsies (N=1,076). The resulting data included genomic profiling data from 2,209 NSCLC biopsies from 1,076 patients.

The oldest and most recent genomic profiles for each patient were paired for further analysis. The median time between biopsies was 439 days and the interquartile range was 202-825 days.

Chromosome arm-level annotations were used to identify chromosome arm level gains or losses (events) as compared to the base ploidy of the sample. Each chromosome arm level event in the genomic profile was analyzed to identify if the event was shared between biopsies of the same patient or unique. Fisher's exact test was used to identify if the chromosome arm level event was more likely to be shared or more likely to be unique between biopsies across the full dataset.

An odds ratio was calculated for each chromosomal event, to determine the uniqueness of the aneuploidy event based on the number of shared and unique occurrences. Events with a greater than 1.5 fold-change in odds ratio in the positive or negative direction were annotated as significantly clonal and subclonal events, respectively.

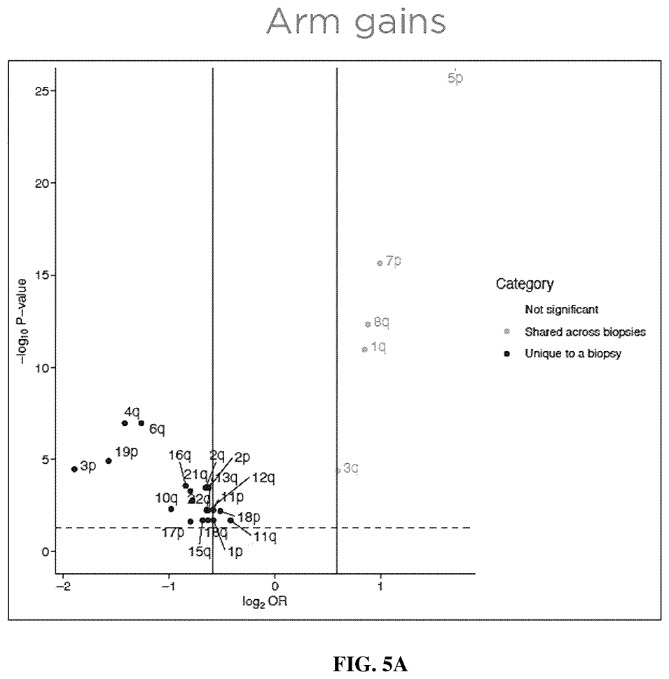

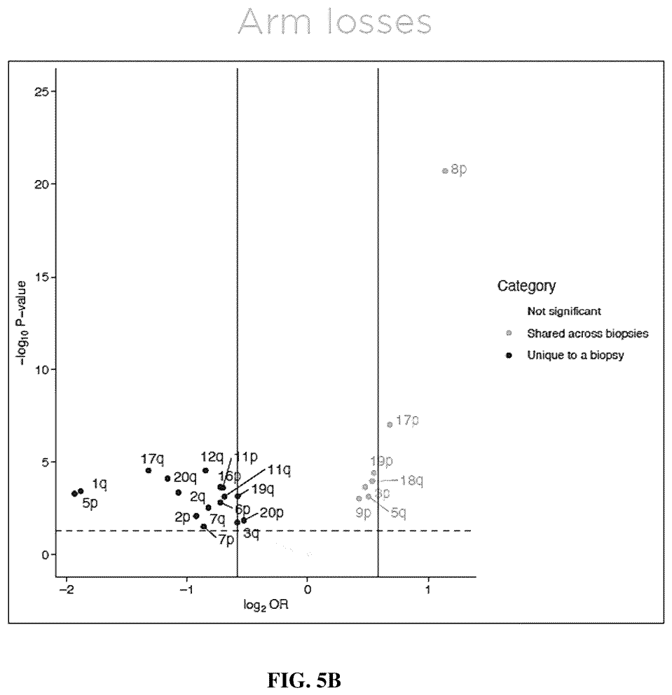

Significantly chromosomal arm level clonal and subclonal gain and losses were identified for NSCLC. FIG. 5A shows the identified clonal and subclonal chromosome arm level gains in the NSCLC dataset. Arm level chromosome gains in 1q, 3q, 5p, 7p and 8q were identified as significantly clonal gains. Arm level chromosome gains, 2p, 2q, 3p, 4q, 6q, 10q, 12q, 13q, 15q, 16q, 17p, 18q, 19p, 21q, and 22q were identified as significantly subclonal gains. FIG. 5B shows the identified clonal and subclonal chromosome arm level losses in the NSCLC dataset. Arm level chromosome losses in 8p and 17p were identified as significantly clonal losses. Arm level chromosome losses, 1q, 2p, 2q, 3q, 5p, 6p, 7p, 7q, 11p, 11q, 12q, 16p, 17q, and 20q were identified as significantly subclonal losses.

### Example 2—Estimation of Aneuploidy Based Intratumor Heterogeneity in all NSCLC Tumor Biopsies

Biopsies from NSCLC patients were examined using a curated list of significantly subclonal chromosome arm level events. The number of significantly subclonal events were used as an indicator of intratumor heterogeneity (ITH).

Comprehensive genomic profiling of DNA extracted from NSCLC patient samples were performed during routine clinical care (N=91,771 NSCLC samples). The comprehensive genomic profile included chromosomal arm-level annotations. The list of significantly subclonal gains and losses described in Example 1 were used to count the number of subclonal events per sample.

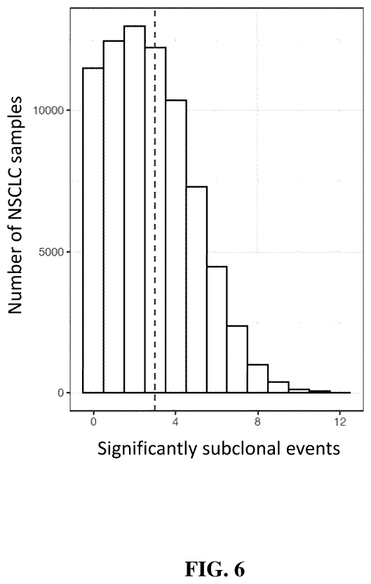

FIG. 6 shows a histogram of the number of NSCLC significantly subclonal events in the 96,705 NSCLC samples. The median number of NSCLC significantly subclonal events per sample was 3 and the interquartile range was 1-4.

An indicator of aneuploidy-based ITH, based on the interquartile range, was assigned to each sample according to the following: low ITH: less than or equal to 1, intermediate ITH: greater than 1 and less than 4, and high ITH greater than or equal to 4.

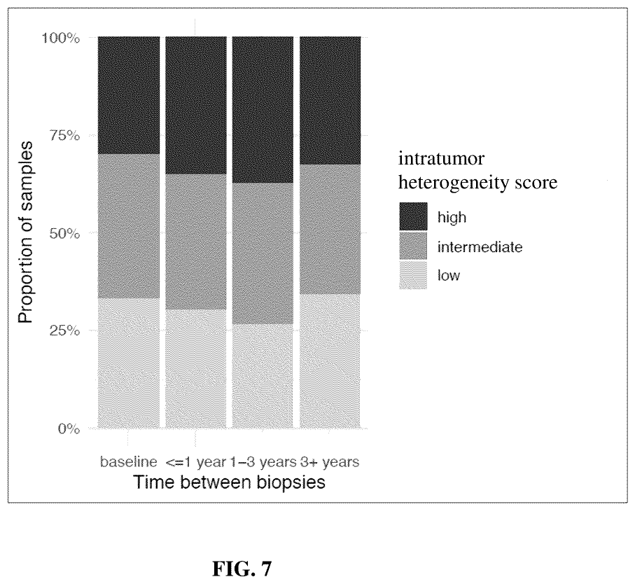

The number of significantly subclonal events were counted and aneuploidy-based ITH scores were assigned to the longitudinal samples described in Example 1. Biopsies were binned according to timepoints, baseline, less than or equal to 1 year after baseline, 1-3 years after baseline, and 3 or more years after baseline, where baseline was the oldest biopsy available for a patient. Results are shown in FIG. 7. The proportion of samples with high ITH increased between baseline, less than or equal to 1 year after baseline, and 1-3 years after baseline.

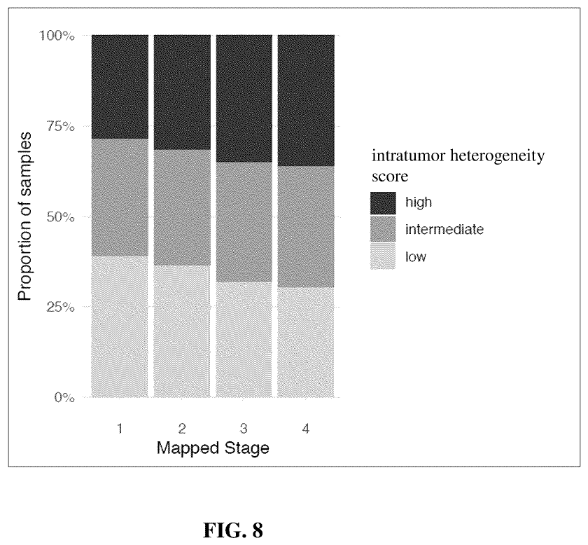

Biopsies were binned according to the mapped stage of the cancer at biopsy (1-4). Results are shown in FIG. 8. The proportion of samples with high ITH increased between cancer stage 1, 2, 3, and 4.

### Example 3—Longitudinal Biopsies to Determine High-Confidence Clonal Vs Subclonal Chromosome Arm Level Events in Ovarian Cancer

Longitudinal tumor biopsies from ovarian cancer patients were examined to identify significantly clonal and subclonal chromosome arm level events. The events were used to test for intratumor heterogeneity (ITH) in ovarian tumor biopsies.

Comprehensive genomic profiling of DNA extracted from ovarian cancer patient samples were performed during routine clinical care (N=20,748). Copy number modeling was used to calculate tumor purity and samples with less than 30% purity were removed from the dataset. The comprehensive genomic profile included chromosomal arm-level annotations. The data set was further filtered to data originating from patients with genomic profiling data for 2 or more biopsies (N=1,126). The resulting data included genomic profiling data from 2,330 ovarian cancer biopsies from 1,126 patients. The oldest and most recent genomic profiles for each patient were paired for further analysis.

Chromosome arm-level annotations were used to identify significant chromosome arm level clonal and subclonal gains or losses (events) as described in Example 1.

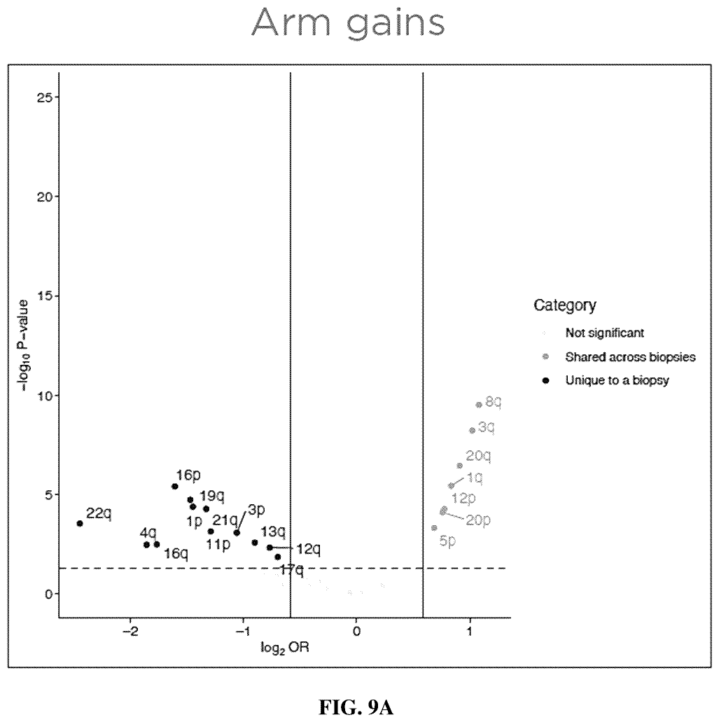

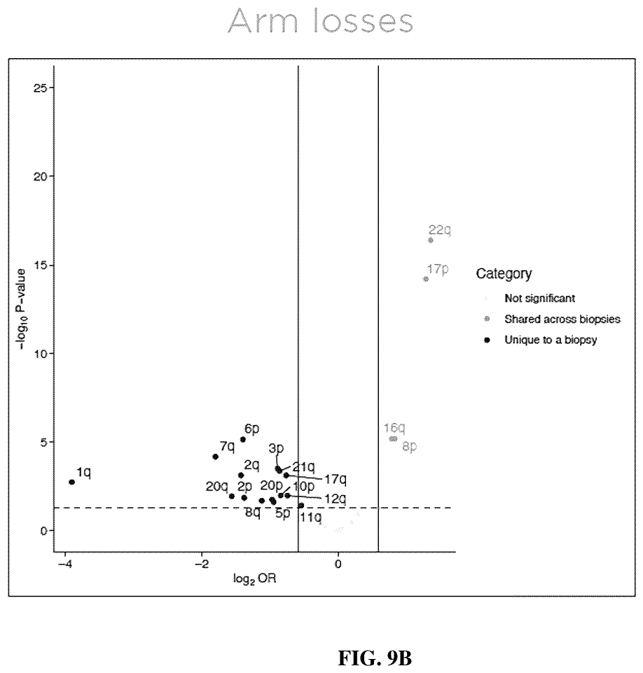

Significant chromosomal arm level clonal and subclonal gain and losses were identified for ovarian cancer. FIG. 9A shows the identified clonal and subclonal chromosome arm level gains in the ovarian cancer dataset. Arm level chromosome gains in 1q, 3q, 5p, 8q, 12p, 20p, and 20q were identified as significantly clonal gains. Arm level chromosome gains, 1p, 3p, 4q, 11p, 12q, 13q, 16p, 16q, 17q, 19q, 21q and 22q, were identified as significantly subclonal gains. FIG. 9B shows the identified clonal and subclonal chromosome arm level losses in the ovarian cancer dataset. Arm level chromosome losses in 8p, 16q, 17p, and 22q were identified as significantly clonal losses. Arm level chromosome losses, 1q, 2p, 2q, 3p, 5p, 6p, 7q, 8q, 10p, 12q, 17q, 20p, 20q, and 21q were identified as significantly subclonal losses.

### Example 4—Estimation of Aneuploidy Based Intratumor Heterogeneity in all Ovarian Tumor Biopsies

Biopsies from ovarian cancer patients were examined using a curated list of significantly subclonal chromosome arm level events. The number of significantly subclonal events was used as an indicator of intratumor heterogeneity (ITH).

Comprehensive genomic profiling of DNA extracted from ovarian cancer patient samples were performed during routine clinical care (N=26,092 ovarian cancer samples). The comprehensive genomic profile included chromosomal arm-level annotations. The list of significantly subclonal gains and losses described in Example 3 were used to count the number of subclonal events per sample.

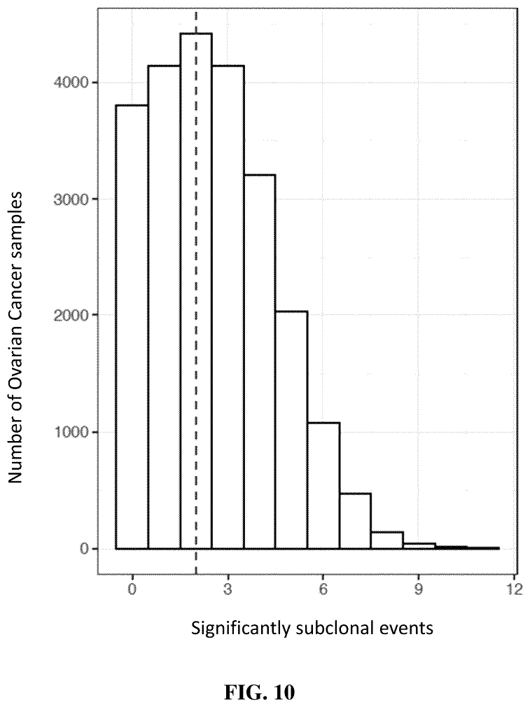

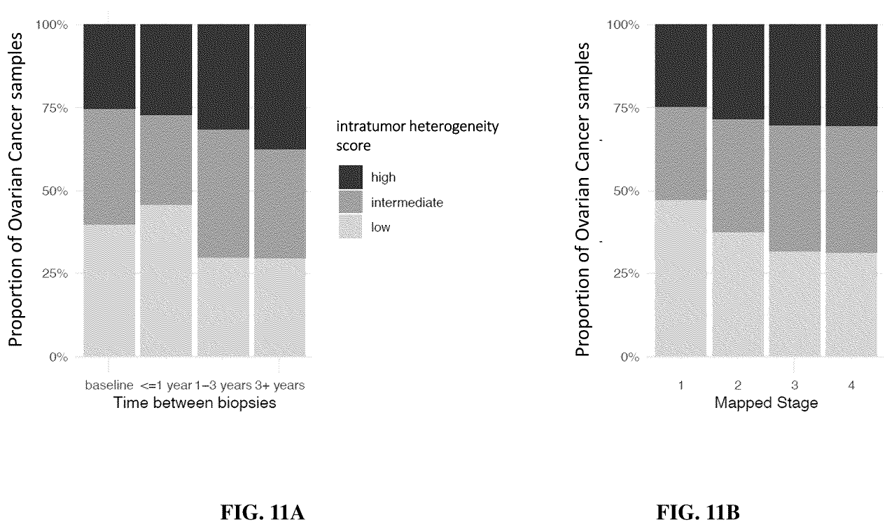

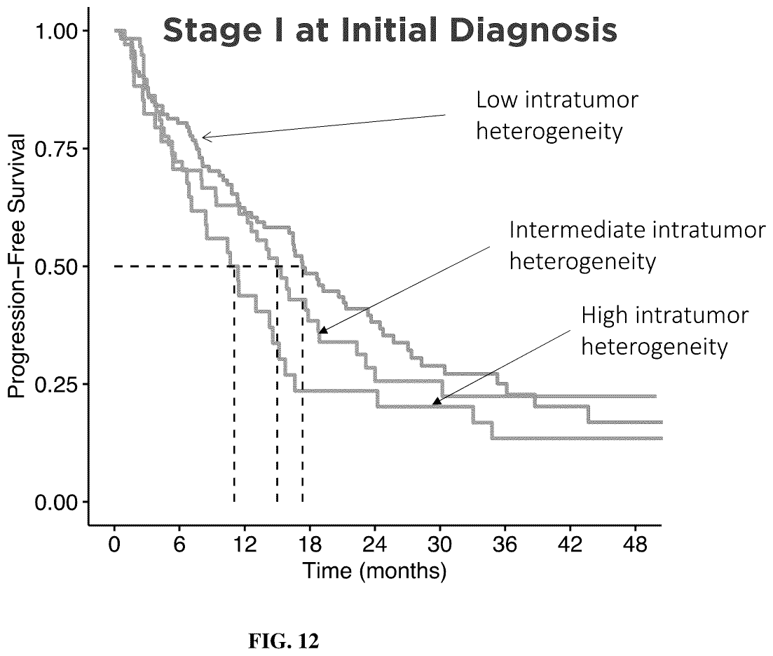

FIG. 10 shows a histogram of the number of ovarian significantly subclonal events in the 26,092 ovarian cancer samples. The median number of ovarian significantly subclonal events per sample was 2 and the interquartile range was 1-4. An indicator of aneuploidy-based ITH, based on the interquartile range, was assigned to each sample according to the following: low ITH: less than or equal to 1, intermediate ITH: greater than 1 and less than 4, and high ITH greater than or equal to 4. The analyses from Example 2 were performed using the ovarian cancer samples. The proportion of samples with high ITH increased according to increasing time differences between baseline and subsequent biopsies (FIG. 11A) and with increases in mapped stage of the cancer (FIG. 11B).

### Example 5—Longitudinal Biopsies to Determine High-Confidence Subclonal Chromosome Arm Level Events in Breast Cancer and Relationship to Survival

Longitudinal tumor biopsies from breast cancer patients were examined to identify significantly clonal and subclonal chromosome arm level events. The events were used to test for intratumor heterogeneity (ITH) in breast tumor biopsies.

This study include breast cancer patients that were diagnosed with stage 1 breast cancer and received CDK4/6 inhibitor and endocrine therapy in the first-line metastatic setting. CDK4/6 inhibitor and endocrine therapy is the first is the standard of care for patients with HR+ HER2− advanced breast cancer, confirmation of the HR+ HER2− status for the breast cancer was not required for inclusion in the analysis. Comprehensive genomic profiling of DNA extracted from samples collected from the breast cancer patients was performed during routine clinical care. The comprehensive genomic profiling was performed on DNA extracted from at least two samples from each patient and for analysis the first and most recent biopsy sample data was used. The comprehensive genomic profile included chromosomal arm-level annotations for each sample.

Chromosome arm-level annotations were used to identify significant chromosome arm level clonal and subclonal gains or losses (events) as described in Example 1.

Significant chromosomal arm level subclonal gain and losses were identified for breast cancer. Arm level chromosome gains, 1p, 2p, 2q, 3p, 4p, 4q, 9q, 10q, 11p, 11q, 13q, 14q, 15q, 16q, 17p, 18p, 18q, 19p, 19q, 21q, and 22q were identified as significantly subclonal gains. Arm level chromosome losses, 1q, 2p, 2q, 3q, 5p, 5p, 7p, 8q, 10p, 16p, 19p, 19q, 20p, and 20q were identified as significantly subclonal loses.

The number of significantly subclonal events identified in each patient was used as an indicator of intratumor heterogeneity (ITH). The median score was 3 with an inter-quartile range of 1-5. An indicator of aneuploidy-based ITH, based on the interquartile range, was assigned to each sample according to the following: low ITH: less than or equal to 1, intermediate ITH: greater than 1 and less than 5, and high ITH greater than or equal to 5.

The patients were assessed for real-world progression-free survival from start of therapy, stratified by their ITH score as determined using a solid tumor biopsy sequenced prior to treatment initiation. Amongst patients who were stage I at initial diagnosis, patients with higher amounts of ITH progressed most quickly. As shown in FIG. 12, patients with the highest level of ITH (34 patients) had the lowest progress-free survival (PFS) at 11.0 months with an interquartile range of 7.1 months to 15.7 months. Patients with intermediate level of ITH (60 patients) had a progress-free survival (PFS) of 15.0 months with 95% confidence interval of 11.5 months to 22.3 months. Patients with a low level of ITH (116 patients) had the highest progress-free survival (PFS) observed at 17.3 months with an 95% confidence interval of 13.8 months to 23.7 months. In comparing the individuals with low ITH and high ITH the hazard ratio was 0.67 with a 95% confidence interval of 0.43 to 1. The p-value associated with the hazard ratio between the low ITH and high ITH groups was p=0.07. Overall, these results suggested that ITH can be used to identify patients with early stage breast cancer who are most at risk for progression.

What is claimed is:

1. A method of characterizing aneuploidy based intratumor heterogeneity for a tumor of a tumor type from a subject, comprising:
obtaining sample aneuploidy data for the tumor;
calling subclonal aneuploidy events in the sample aneuploidy data, wherein a subject aneuploidy event is characterized as subclonal based on a comparison of the subject aneuploidy event to a corresponding reference aneuploidy event for the tumor type, wherein the reference aneuploidy event had been characterized by:
obtaining reference aneuploidy data for a plurality of reference tumor samples of the tumor type, wherein the plurality of reference tumor samples comprises at least two tumor samples obtained at different time points from each reference subject in a plurality of reference subjects,
characterizing, for each aneuploidy event in a plurality of aneuploidy events, as unique or shared among the at least two tumor samples from each reference subject in the plurality of reference subjects,
determining, for each aneuploidy event in the plurality of aneuploidy events, whether uniqueness of the aneuploidy event within the plurality of aneuploid events is enriched compared to uniqueness of all aneuploidy events within the plurality of aneuploidy events, and
characterizing the aneuploidy event as significantly subclonal or significantly clonal based on enrichment of the uniqueness of the aneuploidy event; and
generating an intratumor heterogeneity score for the tumor sample based on a number of called significantly subclonal events in the sample aneuploidy data for the tumor sample.

2. The method of claim 1, wherein the sample aneuploidy data comprises one or more aneuploidy event annotations detected in a single sample collected from the tumor.

3. The method of claim 1, wherein the reference aneuploidy data comprises one or more aneuploidy event annotations for the plurality of reference tumor samples of the tumor type.

4. The method of claim 1, wherein the one or more aneuploidy event annotations are characterized as a variation in chromosome number from a base ploidy of the sample.

5. The method of claim 3, wherein the one or more aneuploidy event annotations comprise a plurality of aneuploidy events.

6. The method of claim 5, wherein an aneuploidy event in the plurality of aneuploidy events is a gain of a chromosomal portion or a loss of a chromosomal portion.

7. The method of claim 1, wherein the tumor type is non-small cell lung cancer (NSCLC), breast cancer, or ovarian cancer.

8. The method of claim 1, wherein determining whether uniqueness of an aneuploidy event within the plurality of aneuploid events is enriched compared to uniqueness of all aneuploidy events within the plurality of aneuploidy events comprises comparing how often the aneuploidy event is subclonal, performing a Fisher's exact test, or performing a chi-squared test.

9. The method of claim 8, wherein performing the Fisher's exact test comprises generating an odds ratio.

10. The method of claim 9, wherein an aneuploidy event is significantly subclonal if the fold change in odds ratio is beyond a cutoff in the negative direction.

11. The method of claim 10, wherein an aneuploidy event is significantly clonal if the fold change in odds ratio beyond a cutoff in the positive direction.

12. The method of claim 1, wherein obtaining sample aneuploidy data comprises; performing a tumor biopsy; extracting tumor nucleic acids; sequencing, by a sequencer, the extracted tumor nucleic acids; receiving a tumor sequence data from the sequencers; and providing the tumor sequence data to a program configured to receive tumor sequence data and identify a plurality of aneuploidy annotations.

13. The method of claim 12, wherein the tumor biopsy is a liquid biopsy and comprises cell-free DNA (cfDNA), circulating tumor DNA (ctDNA), RNA or any combination thereof.

14. The method of claim 1, wherein generating the intratumor heterogeneity score comprises summing the number of called significantly subclonal events in the sample aneuploidy event data for the tumor sample.

15. The method of claim 1, further comprising generating an intratumor heterogeneity indicator, wherein the intratumor heterogeneity indicator relates to the relationship between the intratumor heterogeneity score and a determined threshold.

16. The method of claim 15, wherein the determined threshold comprises an upper threshold and a lower threshold.

17. The method of claim 16, wherein the intratumor heterogeneity indicator is high if the intratumor heterogeneity metric is greater than or equal to the upper threshold, the intratumor heterogeneity indicator is intermediate if the intratumor heterogeneity metric is greater than the lower threshold and less than the lower threshold, and the intratumor heterogeneity indicator is low if intratumor heterogeneity metric is less than or equal to the lower threshold.

18. The method of claim 17, wherein the high intratumor heterogeneity score relates to poor prognosis, quick resistance to cancer therapies, or poor outcomes.

19. The method of claim 1, further comprising generating an aneuploidy burden score by integrating the intratumor heterogeneity score with digital pathology-based heterogeneity, single cell heterogeneity scores, radiological heterogeneity scores, aneuploidy burden, cytoband features, CN segment features for the tumor.

20. A method of treating or delaying progression of cancer in an individual, comprising:
(a) characterizing aneuploidy based intratumor heterogeneity in a sample from the individual according to the methods of claim 1; and
(b) administering to the individual an effective amount of a therapy based on the intratumor heterogeneity.
